(19) United States
(12) Patent Application Publication
(10) Pub. No.: US 2022/0265529 A1
(43) Pub. Date: Aug. 25, 2022

(54) Making An Oral Care Article Of Manufacture

(71) Applicant: The Procter & Gamble Company, Cincinnati, OH (US)

(72) Inventors: Dana Elizabeth Hartsig, West Chester, OH (US); Christopher Robert Lyman, Milford, OH (US); Melissa Cherie Payne, Cincinnati, OH (US); Anthony Edward Reed, West Chester, OH (US); Jeanette Marie Swartz, Loveland, OH (US)

(21) Appl. No.: 17/529,319

(22) Filed: Nov. 18, 2021

Related U.S. Application Data
(63) Continuation of application No. 16/898,468, filed on Jun. 11, 2020, now abandoned.
(60) Provisional application No. 62/860,826, filed on Jun. 13, 2019.

(51) Int. Cl. A61K 8/21; A61K 8/02; A61K 8/19; A61K 8/55; A61Q 11/00; D01D 5/08; D01F 1/10; D01F 8/10; D01F 8/18

(52) U.S. Cl. CPC A61K 8/21; A61K 8/0204; A61K 8/19; A61K 8/55; A61Q 11/00; D01D 5/08; D01F 1/10; D01F 8/10; D01F 8/18

(57) ABSTRACT

A process, for example a continuous process, for making an oral care article of manufacture containing a fibrous composition, for example a composite structure, and more particularly to a process for making an oral care article of manufacture containing a fibrous composition, such as a soluble fibrous composition, containing soluble filaments is provided.

### FIELD OF THE INVENTION

The present invention relates to a process for making an oral care article of manufacture comprising a fibrous composition and a nonfibrous composition. The present invention also relates to a process for making an oral care article of manufacture comprising a fibrous composition, such as a soluble fibrous composition, comprising soluble filaments, and a nonfibrous composition within an internal volume of the oral care article.

### BACKGROUND OF THE INVENTION

Processes for making fibrous compositions, for example soluble fibrous compositions, and/or components thereof, such as soluble filaments, are known in the art. However, such known processes to date have been discontinuous. In other words, such known processes have at least two or more discrete (discontinuous) steps or unit operations that interrupt the process of making an oral care article of manufacture, for example one or more steps of making a fibrous composition uncoupled and/or discrete from one or more steps of converting the made fibrous composition into the article of manufacture, for example a consumer product. Such a non-continuous/discontinuous process may comprise one or more of the following steps: 1) a filament-forming composition making step, such as a batch process to make a filament-forming composition; 2) a spinning step for spinning the filament-forming composition to make filaments, for example soluble filaments; 3) optionally, a commingling (coforming) step for commingling solid additives, for example particles, with filaments; 4) a collection step for collecting the filaments and/or commingled filaments and solid additives on a collection device to form a fibrous composition, for example a soluble fibrous composition; 5) a converting operation (one or more steps for converting (for example slitting and/or stacking and/or calendering and/or treating with a nonfibrous composition, such as a liquid, paste, or solid composition comprising an abrasive, whitening agents, flavoring agents, effervescent agents, and the like, die-cutting, and printing) the fibrous composition into one or more articles of manufacture, for example a consumer product); and 6) optionally a packaging step for packaging the articles of manufacture.

One problem faced by formulators is how to make such articles of manufacture comprising fibrous compositions, for example soluble fibrous compositions, in a continuous or more continuous than the known discontinuous process. In other words, one problem faced by formulators is how to combine multiple process steps from above into a continuous process such that they are not discrete, discontinuous process steps.

Additionally, oral care articles of manufacture comprising dentifrice compositions can comprise components, such as abrasives, polyphosphates, flavors, or whitening agents, that are unable to survive the spinning step because they can clog the spinning die or undergo degradation at high temperatures.

Accordingly, there is a need for a process for making oral care articles of manufacture, for example consumer products, comprising a fibrous composition, for example a soluble fibrous composition, and a nonfibrous composition, in a continuous or at least partially continuous process.

### SUMMARY OF THE INVENTION

The present invention fulfills the need described above by providing a continuous process and/or continuous process steps within the process to make an oral care article of manufacture, for example a consumer product, comprising a fibrous composition, for example a soluble fibrous composition.

Additionally, the present invention fulfills the need described above by creating a depression in the fibrous composition for the placement of a nonfibrous composition comprising one or more oral care active agents that are unable to undergo the spinning step.

The present invention discloses a process for making a fibrous structure, the process comprising the steps of (a) providing one or more soluble filament-forming materials, (b) forming an aqueous composition comprising the one or more soluble filament-forming materials (c) processing the aqueous composition to produce a filament-forming composition, (d) delivering the filament-forming composition to one or more dies, (e) spinning the filament-forming composition to form a plurality of soluble filaments (f) collecting the soluble filaments on a collection device to form a fibrous structure, (g) applying pressure to the fibrous structure to form a depression in the fibrous structure, and optionally (h) adding a nonfibrous composition to the depression in the fibrous structure.

The present invention provides a continuous process for making a fibrous composition and ultimately an oral care article of manufacture.

### BRIEF DESCRIPTION OF THE DRAWINGS

FIG. 1 is a schematic representation of an example of a process according to the present invention;

FIG. 2 is a schematic representation of an example of a portion of the process according to the present invention;

FIG. 3 is a schematic representation of an example of an extruder screw suitable for use in the process according to the present invention;

FIG. 4 is a schematic representation of an example of a portion of the process according to the present invention;

FIG. 5 is a top plan view of a die suitable for use in the process according to the present invention;

FIG. 6 is a schematic representation of an example of a portion of the process according to the present invention;

FIG. 7 is a schematic representation of an example of a formation zone on a collection device suitable for use in the process according to the present invention;

FIG. 8 is a schematic representation of an example of a process according to the present invention; and

FIG. 9 is a schematic representation of an example of a portion of the process according to the present invention.

### DETAILED DESCRIPTION OF THE INVENTION

### Definitions

“Fibrous composition” as used herein means a structure that comprises one or more filaments and optionally, one or more particles. In one example, a fibrous composition according to the present invention means an association of filaments and optionally, particles that together form a structure, such as a unitary structure, capable of performing a function.

The fibrous compositions of the present invention may be single layered or multi-layered. If multi-layered, the fibrous compositions may comprise at least two and/or at least three and/or at least four and/or at least five layers and/or at least six layers, for example one or more filament layers, one or more particle layers and/or one or more composite structure layers having a mixture of filaments and particles. A layer may comprise a particle layer within the fibrous composition or between filament layers within a fibrous composition. A layer comprising filaments may sometimes be referred to as a ply. A ply may be a fibrous composition which may be single layered or multi-layered as described herein. In one example, a layer may be formed by a single spinning die and/or particle delivery source or if it is a composite structure layer, then is may be formed by a single spinning die and a particle delivery source.

In one example, the fibrous compositions of the present invention may comprise single or multiple layers, at least one of which must comprise fibers. Layers may include additives (for example, pastes or sprays) applied to said fibers and/or particles comingled with said fibers in a composite structure.

In one example, a single-ply fibrous composition according to the present invention or a multi-ply fibrous composition comprising one or more fibrous composition plies according to the present invention may exhibit a basis weight of less than 5000 g/m² as measured according to the Basis Weight Test Method described herein. In one example, the single- or multi-ply fibrous composition according to the present invention may exhibit a basis weight of greater than 10 g/m² to about 5000 g/m² and/or greater than 10 g/m² to about 3000 g/m² and/or greater than 10 g/m² to about 2000 g/m² and/or greater than 10 g/m² to about 1000 g/m² and/or greater than 20 g/m² to about 800 g/m² and/or greater than 30 g/m² to about 600 g/m² and/or greater than 50 g/m² to about 500 g/m² and/or greater than 300 g/m² to about 3000 g/m² and/or greater than 500 g/m² to about 2000 g/m² as measured according to the Basis Weight Test Method.

In one example, a single ply comprising a multi-layered fibrous composition comprises a first layer, such as a scrim layer comprising a plurality of filaments present at a basis weight of from about 10 to about 200 gsm and/or from about 30 to about 100 gsm and/or from about 50 to about 75 gsm and a second layer, for example a layer comprising a plurality of filaments, alone or as a composite structure layer comprising filaments and solid additives, for example particles, present at a basis weight of from about 400 to about 3000 gsm and/or from about 600 to about 1500 gsm and/or from about 800 to about 1200 gsm.

In one example, the fibrous composition of the present invention is a “unitary fibrous composition.”

“Unitary fibrous composition” as used herein is an arrangement comprising a plurality of two or more and/or three or more filaments that are inter-entangled or otherwise associated with one another to form a fibrous composition and/or fibrous composition plies. A unitary fibrous composition of the present invention may be one or more plies within a multi-ply fibrous composition. In one example, a unitary fibrous composition of the present invention may comprise three or more different filaments. In another example, a unitary fibrous composition of the present invention may comprise two or more different filaments.

“Nonfibrous composition,” as used herein refers to a composition that is substantially free of or free of fibers and/or filaments. The nonfibrous composition can be a solid, semisolid, semiliquid, liquid, aqueous solution, or combinations thereof. The oral care article can comprise a nonfibrous composition, which may or may not be greater in weight percentage, by weight of the oral care article, than the fibrous composition. The nonfibrous composition can be between a first fibrous composition and a second fibrous composition. At least a portion of the nonfibrous composition can be in contact with a surface of fibrous composition. The nonfibrous composition can be placed on a single fibrous composition and the fibrous composition can be folded on top of the nonfibrous composition, rolled with the nonfibrous composition, placed on top of or below the fibrous composition, and/or the fibrous composition can wrap around the fibrous composition.

The nonfibrous composition can comprise any suitable oral care active agent. The nonfibrous composition can comprise any component described herein. The nonfibrous composition can be liquid, solid, aqueous, and/or combinations thereof.

The components described herein can optionally be present, at least partially, as a nonfibrous composition. The nonfibrous composition can be between two or more web layers, folded inside at least one web layer, rolled inside at least web layer, or wrapped in at least one web layer. At least a portion of the nonfibrous composition can contact the surface of a fibrous composition. The nonfibrous composition can be liquid, solid, aqueous, and/or combinations thereof.

The nonfibrous composition may comprise an oral care active, aesthetic agent, abrasive, fluoride ion source, web forming material, metal ion source, polyphosphate, chelant, anti-calculus agent, thickening agent, polymer, surfactant, bioactive material and/or combinations thereof. The nonfibrous composition can be from about 10% to about 90%, from about 20% to about 85%, from about 30% to about 80%, from about 40% to about 75%, from about 50% to about 80%, from about 50% to about 90%, or from about 60% to about 80% by weight of the oral care composition.

The density of the nonfibrous composition can be from about 0.05 g/cm³ to about 5 g/cm³, from about 0.75 g/cm³ to about 1.9 g/cm³, from about 1 g/cm³ to about 1.75 g/cm³, or from about 1.4 g/cm³ to about 1.8 g/cm3.

“Article” as used herein refers to a consumer use unit, a consumer unit dose unit, a consumer use saleable unit, a single dose unit, or other use form comprising a unitary fibrous composition and/or comprising one or more fibrous compositions of the present invention.

“Fibrous element” as used herein means an elongate particulate having a length greatly exceeding its average diameter, i.e. a length to average diameter ratio of at least about 10. A fibrous element may be a filament or a fiber. In one example, the fibrous element is a single filament rather than a yarn comprising a plurality of filaments.

The fibrous elements of the present invention may be spun from fibrous element-forming compositions also referred to as filament-forming compositions via suitable spinning process operations, such as meltblowing, spunbonding, electro-spinning, and/or rotary spinning

The fibrous elements of the present invention may be monocomponent (single, unitary solid piece rather than two different parts, like a core/sheath bicomponent) and/or multicomponent. For example, the fibrous elements may comprise bicomponent fibers and/or filaments. The bicomponent fibers and/or filaments may be in any form, such as side-by-side, core and sheath, islands-in-the-sea and the like.

“Filament” as used herein means an elongate particulate as described above that exhibits a length of greater than or equal to 5.08 cm (2 in.) and/or greater than or equal to 7.62 cm (3 in.) and/or greater than or equal to 10.16 cm (4 in.) and/or greater than or equal to 15.24 cm (6 in.).

Filaments are typically considered continuous or substantially continuous in nature. Filaments are relatively longer than fibers. Non-limiting examples of filaments include meltblown and/or spunbond filaments. Non-limiting examples of polymers that can be spun into filaments include natural polymers, such as starch, starch derivatives, cellulose, such as rayon and/or lyocell, and cellulose derivatives, hemicellulose, hemicellulose derivatives, and synthetic polymers including, but not limited to polyvinyl alcohol and also thermoplastic polymer filaments, such as polyesters, nylons, polyolefins such as polypropylene filaments, polyethylene filaments, and biodegradable thermoplastic fibers such as polylactic acid filaments, polyhydroxyalkanoate filaments, polyesteramide filaments and polycaprolactone filaments.

“Fiber” as used herein means an elongate particulate as described above that exhibits a length of less than 5.08 cm (2 in.) and/or less than 3.81 cm (1.5 in.) and/or less than 2.54 cm (1 in.).

Fibers are typically considered discontinuous in nature. Non-limiting examples of fibers include staple fibers produced by spinning a filament or filament tow of the present invention and then cutting the filament or filament tow into segments of less than 5.08 cm (2 in.) thus producing fibers.

In one example, one or more fibers may be formed from a filament of the present invention, such as when the filaments are cut to shorter lengths (such as less than 5.08 cm in length). Thus, in one example, the present invention also includes a fiber made from a filament of the present invention, such as a fiber comprising one or more filament-forming materials and one or more fiber adjuncts, such as active agents. Therefore, references to filament and/or filaments of the present invention herein also include fibers made from such filament and/or filaments unless otherwise noted. Fibers are typically considered discontinuous in nature relative to filaments, which are considered continuous in nature.

“Fibrous element-forming composition” and/or “filament-forming composition” as used herein means a composition that is suitable for making a filament of the present invention such as by meltblowing and/or spunbonding. The filament-forming composition comprises one or more filament-forming materials that exhibit properties that make them suitable for spinning into a filament. In one example, the filament-forming material comprises a polymer. In addition to one or more filament-forming materials, the filament-forming composition may comprise one or morefiber adjuncts, for example one or more active agents. In addition, the filament-forming composition may comprise one or more polar solvents, such as water, into which one or more, for example all, of the filament-forming materials and/or one or more, for example all, of the active agents are dissolved and/or dispersed prior to spinning a filament, such as a filament from the filament-forming composition.

In one example, a filament made from a filament-forming composition of the present invention is such that one or more fiber adjuncts, for example one or more active agents, may be present in the filament rather than on the filament, such as a coating composition comprising one or more active agents, which may be the same or different from the active agents in the filaments and/or particles. The total level of filament-forming materials and total level of active agents present in the filament-forming composition may be any suitable amount so long as the filaments of the present invention are produced therefrom.

In one example, one or more fiber adjuncts, such as active agents, may be present in the filament and one or more additional fiber adjuncts, such as active agents, may be present on a surface of the filament. In another example, a filament of the present invention may comprise one or more fiber adjuncts, such as active agents, that are present in the filament when originally made, but then bloom to a surface of the filament prior to and/or when exposed to conditions of intended use of the filament.

“Fibrous element-forming material” and/or “filament-forming material” as used herein means a material, such as a polymer or monomers capable of producing a polymer that exhibits properties suitable for making a filament. In one example, the filament-forming material comprises one or more substituted polymers such as an anionic, cationic, zwitterionic, and/or nonionic polymer. In another example, the polymer may comprise a hydroxyl polymer, such as a polyvinyl alcohol (“PVOH”), a partially hydrolyzed polyvinyl acetate and/or a polysaccharide, such as starch and/or a starch derivative, such as an ethoxylated starch and/or acid-thinned starch, carboxymethylcellulose, hydroxypropyl cellulose, hydroxyethyl cellulose, and methyl cellulose. In another example, the polymer may comprise polyethylenes and/or terephthalates. In yet another example, the filament-forming material is a polar solvent-soluble material.

“Particle” as used herein means a solid additive, such as a powder, granule, agglomerate, encapsulate, microcapsule, and/or prill. The shape of the particle can be in the form of spheres, rods, plates, tubes, squares, rectangles, discs, stars, fibers or have regular or irregular random forms. The particles of the present invention, at least those of at least 44 μm, can be measured by the Particle Size Distribution Test Method described herein. For particles that are less than 44 μm, a different test method may be used, for example light scattering, to determine the particle sizes less than 44 μm, for example perfume microcapsules that typically range from about 15 μm to about 44 μm and/or about 25 μm in size.

In one aspect, particles may comprise re-cycled fibrous-structure materials, specifically where said fibrous materials are re-cycled by grinding fibers into a finely-divided solid and re-incorporating said finely-divided solids into agglomerates, granules or other particle forms. In another aspect, particles may comprise re-cycled fibrous-structure materials, specifically where said fibrous materials are incorporated into a fluid paste, suspension or solution, and then processed to form agglomerates, granules or other particle forms. In another aspect, said fluid pastes, suspensions or solutions comprising recycled fibrous materials may be directly applied to fibrous layers in the process of making new fibrous articles.

“Active agent-containing particle” as used herein means a solid additive, for example a particle, comprising one or more active agents. In one example, the active agent-containing particle is an active agent in the form of a particle (in other words, the particle comprises 100% active agent(s)). The active agent-containing particle may exhibit a particle size of 5000 μm or less as measured according to the Particle Size Distribution Test Method described herein.

In one example of the present invention, the fibrous composition comprises a plurality of particles, for example active agent-containing particles, and a plurality of filaments in a weight ratio of particles, for example active agent-containing particles to filaments of 1:100 or greater and/or 1:50 or greater and/or 1:10 or greater and/or 1:3 or greater and/or 1:2 or greater and/or 1:1 or greater and/or 2:1 or greater and/or 3:1 or greater and/or 4:1 or greater and/or 5:1 or greater and/or 7:1 or greater and/or 8:1 or greater and/or 10:1 or greater and/or from about 10:1 to about 1:100 and/or from about 8:1 to about 1:50 and/or from about 7:1 to about 1:10 and/or from about 7:1 to about 1:3 and/or from about 6:1 to 1:2 and/or from about 5:1 to about 1:1 and/or from about 4:1 to about 1:1 and/or from about 3:1 to about 1.5:1.

In another example of the present invention, the fibrous composition comprises a plurality of particles, for example active agent-containing particles, and a plurality of filaments in a weight ratio of particles, for example active agent-containing particles, to filaments of from about 20:1 to about 1:1 and/or from about 10:1 to about 1:1 and/or from about 10:1 to about 1.5:1 and/or from about 8:1 to about 1.5:1 and/or from about 8:1 to about 2:1 and/or from about 7:1 to about 2:1 and/or from about 7:1 to about 3:1 and/or from about 6:1 to about 2.5:1.

In yet another example of the present invention, the fibrous composition comprises a plurality of particles, for example active agent-containing particles, and a plurality of filaments in a weight ratio of particles, for example active agent-containing particles, to filaments of from about 1:1 to about 1:100 and/or from about 1:15 to about 1:80, and/or from about 1:2 to about 1:60 and/or from about 1:3 to about 1:50 and/or from about 1:3 to about 1:40.

In another example, the fibrous composition of the present invention comprises a plurality of particles, for example active agent-containing particles, at a basis weight of greater than 1 g/m² and/or greater than 10 g/m² and/or greater than 20 g/m² and/or greater than 30 g/m² and/or greater than 40 g/m² and/or from about 1 g/m² to about 5000 g/m² and/or to about 3500 g/m² and/or to about 2000 g/m² and/or from about 1 g/m² to about 2000 g/m² and/or from about 10 g/m² to about 1000 g/m² and/or from about 10 g/m² to about 500 g/m² and/or from about 20 g/m² to about 400 g/m² and/or from about 30 g/m² to about 300 g/m² and/or from about 40 g/m² to about 200 g/m² as measured by the Basis Weight Test Method described herein.

In another example, the fibrous composition of the present invention comprises a plurality of filaments at a basis weight of greater than 1 g/m² and/or greater than 10 g/m² and/or greater than 20 g/m² and/or greater than 30 g/m² and/or greater than 40 g/m² and/or from about 1 g/m² to about 3000 g/m² and/or from about 10 g/m² to about 5000 g/m² and/or to about 3000 g/m² and/or to about 2000 g/m² and/or from about 20 g/m² to about 2000 g/m² and/or from about 30 g/m² to about 1000 g/m² and/or from about 30 g/m² to about 500 g/m² and/or from about 30 g/m² to about 300 g/m² and/or from about 40 g/m² to about 100 g/m² and/or from about 40 g/m² to about 80 g/m² as measured by the Basis Weight Test Method described herein. In one example, the fibrous composition comprises two or more layers wherein filaments are present in at least one of the layers at a basis weight of from about 1 g/m² to about 500 g/m².

“Commingled” and/or “commingling” as used herein means the state or form where particles are mixed with fibrous elements, for example filaments. The mixture of filaments and particles can be throughout a composite structure or within a plane or a region of the composite structure. In one example, the commingled filaments and particles may form at least a surface of a composite structure. In one example, the particles may be homogeneously dispersed throughout the composite structure and/or plane and/or region of the composite structure. In one example, the particles may be homogeneously distributed throughout the composite structure, which avoids and/or prevents sag and/or free movement and/or migration of the particles within the composite structure to other areas within the composite structure thus resulting in higher concentrated zones of particles and lower concentrated zones or zero concentration zones of particles within the composite structure. In one example, μCT cross-sections of a composite structure can show whether the particles are homogeneously distributed throughout a composite structure.

“Fiber adjunct” as used herein means any material present in the filament of the present invention that is not a filament-forming material. In one example, a fiber adjunct comprises an active agent. In another example, a fiber adjunct comprises a processing aid. In still another example, a fiber adjunct comprises a filler. In one example, a fiber adjunct comprises any material present in the filament that its absence from the filament would not result in the filament losing its filament structure, in other words, its absence does not result in the filament losing its solid form. In another example, a fiber adjunct, for example an active agent, comprises a non-polymer material.

In another example, a fiber adjunct may comprise a plasticizer for the filament. Non-limiting examples of suitable plasticizers for the present invention include polyols, copolyols, polycarboxylic acids, polyesters and dimethicone copolyols. Examples of useful polyols include, but are not limited to, glycerin, diglycerin, propylene glycol, ethylene glycol, butylene glycol, pentylene glycol, cyclohexane dimethanol, hexanediol, 2,2,4-trimethylpentane-1,3-diol, polyethylene glycol (200-600), pentaerythritol, sugar alcohols such as sorbitol, manitol, lactitol and other mono- and polyhydric low molecular weight alcohols (e.g., C2-C8 alcohols); mono di- and oligo-saccharides such as fructose, glucose, sucrose, maltose, lactose, high fructose corn syrup solids, and dextrins, and ascorbic acid.

In one example, the plasticizer includes glycerin and/or propylene glycol and/or glycerol derivatives such as propoxylated glycerol. In still another example, the plasticizer is selected from the group consisting of glycerin, ethylene glycol, polyethylene glycol, propylene glycol, glycidol, urea, sorbitol, xylitol, maltitol, sugars, ethylene bisformamide, amino acids, and mixtures thereof

In another example, a fiber adjunct may comprise a rheology modifier, such as a shear modifier and/or an extensional modifier. Non-limiting examples of rheology modifiers include but not limited to polyacrylamide, polyurethanes and polyacrylates that may be used in the filaments of the present invention. Non-limiting examples of rheology modifiers are commercially available from The Dow Chemical Company (Midland, Mich.).

In yet another example, a fiber adjunct may comprise one or more colors and/or dyes that are incorporated into the filaments of the present invention to provide a visual signal when the filaments are exposed to conditions of intended use and/or when an active agent is released from the filaments and/or when the filament's morphology changes.

In still yet another example, a fiber adjunct may comprise one or more release agents and/or lubricants. Non-limiting examples of suitable release agents and/or lubricants include fatty acids, fatty acid salts, fatty alcohols, fatty esters, sulfonated fatty acid esters, fatty amine acetates, fatty amide, silicones, aminosilicones, fluoropolymers, and mixtures thereof. In one example, the release agents and/or lubricants may be applied to the filament, in other words, after the filament is formed. In one example, one or more release agents/lubricants may be applied to the filament prior to collecting the filaments on a collection device to form a fibrous composition. In another example, one or more release agents/lubricants may be applied to a fibrous composition formed from the filaments of the present invention prior to contacting one or more fibrous compositions, such as in a stack of fibrous compositions. In yet another example, one or more release agents/lubricants may be applied to the filament of the present invention and/or fibrous composition comprising the filament prior to the filament and/or fibrous composition contacting a surface, such as a surface of equipment used in a processing system so as to facilitate removal of the filament and/or fibrous composition and/or to avoid layers of filaments and/or plies of fibrous compositions of the present invention sticking to one another, even inadvertently. In one example, the release agents/lubricants comprise particulates.

In even still yet another example, a fiber adjunct may comprise one or more anti-blocking and/or detackifying agents. Non-limiting examples of suitable anti-blocking and/or detackifying agents include starches, starch derivatives, crosslinked polyvinylpyrrolidone, crosslinked cellulose, microcrystalline cellulose, silica, metallic oxides, calcium carbonate, talc, mica, and mixtures thereof.

“Conditions of intended use” as used herein means the temperature, physical, chemical, and/or mechanical conditions that a filament and/or particle and/or fibrous composition of the present invention is exposed to when the filament and/or particle and/or fibrous composition is used for one or more of its designed purposes. For example, if a filament and/or a particle and/or a fibrous composition comprising a filament is designed to be used in a washing machine for laundry care purposes, the conditions of intended use will include those temperature, chemical, physical and/or mechanical conditions present in a washing machine, including any wash water, during a laundry washing operation. In another example, if a filament and/or a particle and/or a fibrous composition comprising a filament is designed to be used by a human as a shampoo for hair care purposes, the conditions of intended use will include those temperature, chemical, physical and/or mechanical conditions present during the shampooing of the human's hair. Likewise, if a filament and/or a particle and/or a fibrous composition comprising a filament is designed to be used in a dishwashing operation, by hand or by a dishwashing machine, the conditions of intended use will include the temperature, chemical, physical and/or mechanical conditions present in a dishwashing water and/or dishwashing machine, during the dishwashing operation.

“Active agent” as used herein means a fiber adjunct that produces an intended effect in an environment external to a filament and/or a particle and/or a fibrous composition comprising a filament of the present invention, such as when the filament and/or a particle and/or fibrous composition is exposed to conditions of intended use of the filament and/or a particle and/or a fibrous composition comprising a filament. In one example, an active agent comprises a fiber adjunct that treats a surface, such as a hard surface (i.e., kitchen countertops, bath tubs, toilets, toilet bowls, sinks, floors, walls, teeth, cars, windows, mirrors, dishes) and/or a soft surface (i.e., fabric, hair, skin, carpet, crops, plants,). In another example, an active agent comprises additive fiber adjunct that creates a chemical reaction (i.e., foaming, fizzing, effervescing, coloring, warming, cooling, lathering, disinfecting and/or clarifying and/or chlorinating, such as in clarifying water and/or disinfecting water and/or chlorinating water). In yet another example, an active agent comprises a fiber adjunct that treats an environment (i.e., deodorizes, purifies, perfumes air). In one example, the active agent is formed in situ, such as during the formation of the filament and/or particle containing the active agent, for example the filament and/or particle may comprise a water-soluble polymer (e.g., starch) and a surfactant (e.g., anionic surfactant), which may create a polymer complex or coacervate that functions as the active agent used to treat fabric surfaces.

“Treats” as used herein with respect to treating a surface means that the active agent provides a benefit to a surface or environment. Treats includes regulating and/or immediately improving a surface's or environment's appearance, cleanliness, smell, purity and/or feel. In one example treating in reference to treating a keratinous tissue (for example skin and/or hair) surface means regulating and/or immediately improving the keratinous tissue's cosmetic appearance and/or feel. For instance, “regulating skin, hair, or nail (keratinous tissue) condition” includes: thickening of skin, hair, or nails (e.g, building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair. Treats may include providing a benefit to fabrics like during a cleaning or softening in a laundry machine, providing a benefit to hair like during shampooing, conditioning, or coloring of hair, or providing a benefit to environments like a toilet bowl by cleaning or disinfecting it.

In another example, treating means removing stains and/or odors from fabric articles, such as clothes, towels, linens, and/or hard surfaces, such as countertops and/or dishware including pots and pans.

“Fabric care active agent” as used herein means an active agent that when applied to a fabric provides a benefit and/or improvement to the fabric. Non-limiting examples of benefits and/or improvements to a fabric include cleaning (for example by surfactants), stain removal, stain reduction, wrinkle removal, color restoration, static control, wrinkle resistance, permanent press, wear reduction, wear resistance, pill removal, pill resistance, soil removal, soil resistance (including soil release), shape retention, shrinkage reduction, softness, fragrance, anti-bacterial, anti-viral, odor resistance, and odor removal.

“Dishwashing active agent” as used herein means an active agent that when applied to dishware, glassware, pots, pans, utensils, and/or cooking sheets provides a benefit and/or improvement to the dishware, glassware, plastic items, pots, pans and/or cooking sheets. Non-limiting examples of benefits and/or improvements to the dishware, glassware, plastic items, pots, pans, utensils, and/or cooking sheets include food and/or soil removal, cleaning (for example by surfactants) stain removal, stain reduction, grease removal, water spot removal and/or water spot prevention, glass and metal care, sanitization, shining, and polishing.

“Hard surface active agent” as used herein means an active agent when applied to floors, countertops, sinks, windows, mirrors, showers, baths, and/or toilets provides a benefit and/or improvement to the floors, countertops, sinks, windows, mirrors, showers, baths, and/or toilets. Non-limiting examples of benefits and/or improvements to the floors, countertops, sinks, windows, mirrors, showers, baths, and/or toilets include food and/or soil removal, cleaning (for example by surfactants), stain removal, stain reduction, grease removal, water spot removal and/or water spot prevention, limescale removal, disinfection, shining, polishing, and freshening.

“Keratinous tissue active agent” as used herein means an active agent that may be useful for treating keratinous tissue (e.g., hair, skin, or nails) condition. For a hair care active agent, “treating” or “treatment” or “treat” includes regulating and/or immediately improving keratinous tissue cosmetic appearance and/or feel. For instance, “regulating skin, hair, or nail condition” includes: thickening of skin, hair, or nails (e.g., building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair. Another example of keratinous tissue active agent may be an active agent used in the shampooing, conditioning, or dyeing of hair.

“Weight ratio” as used herein means the ratio between two materials on their dry basis. For example, the weight ratio of filament-forming materials to active agents within a filament is the ratio of the weight of filament-forming material on a dry weight basis (g or %) in the filament to the weight of fiber adjunct, such as active agent(s) on a dry weight basis (g or %—same units as the filament-forming material weight) in the filament. In another example, the weight ratio of particles to filaments within a fibrous composition is the ratio of the weight of particles on a dry weight basis (g or %) in the fibrous composition to the weight of filaments on a dry weight basis (g or %—same units as the particle weight) in the fibrous composition.

“Water-soluble material” as used herein means a material that is miscible in water. In other words, a material that is capable of forming a stable (does not separate for greater than 5 minutes after forming the homogeneous solution) homogeneous solution with water at ambient conditions. “Ambient conditions” as used herein means 23° C.±1.0° C. and a relative humidity of 50%±2%.

“Weight average molecular weight” as used herein means the weight average molecular weight as determined using gel permeation chromatography according to the protocol found in Colloids and Surfaces A. Physico Chemical & Engineering Aspects, Vol. 162, 2000, pg. 107-121.

“Length” as used herein, with respect to a filament, means the length along the longest axis of the filament from one terminus to the other terminus. If a filament has a kink, curl or curves in it, then the length is the length along the entire path of the filament from one terminus to the other terminus.

“Diameter” as used herein, with respect to a filament, is measured according to the Diameter Test Method described herein. In one example, a filament of the present invention exhibits a diameter of less than 100 μm and/or less than 75 μm and/or less than 50 μm and/or less than 25 μm and/or less than 20 μm and/or less than 15 μm and/or less than 10 μm and/or less than 6 μm and/or greater than 1 μm and/or greater than 3 μm.

“Triggering condition” as used herein means anything, as an act or event that serves as a stimulus and initiates or precipitates a change in the filament, such as a loss or altering of the filament's physical structure and/or a release an oral care active including dissolution, hydration, and swelling. Some triggering conditions include a suitable pH, temperature, shear rate, or water content.

“Morphology changes” as used herein with respect to a filament's morphology changing means that the filament experiences a change in its physical structure. Non-limiting examples of morphology changes for a filament of the present invention include dissolution, melting, swelling, shrinking, breaking into pieces, lengthening, shortening, peeling, splitting, shredding, imploding, twisting, and combinations thereof. The filaments of the present invention may completely or substantially lose their filament physical structure or they may have their morphology changed or they may retain or substantially retain their filament physical structure as they are exposed to conditions of intended use.

“By weight on a dry filament basis” and/or “by weight on a dry particle basis” and/or “by weight on a dry fibrous composition basis” means the weight of the filament and/or particle and/or fibrous composition, respectively, measured immediately after the filament and/or particle and/or fibrous composition, respectively, has been conditioned in a conditioned room at a temperature of 23° C.±1.0° C. and a relative humidity of 50%±10% for 2 hours. In one example, by weight on a dry filament basis and/or dry particle basis and/or dry fibrous composition basis means that the filament and/or particle and/or fibrous composition comprises less than 20% and/or less than 15% and/or less than 10% and/or less than 7% and/or less than 5% and/or less than 3% and/or to 0% and/or to greater than 0% based on the dry weight of the filament and/or particle and/or fibrous composition of moisture, such as water, for example free water, as measured according to the Water Content Test Method described herein.

“Total level” as used herein, for example with respect to the total level of one or more active agents present in the filament and/or particle and/or fibrous composition, means the sum of the weights or weight percent of all of the subject materials, for example active agents. In other words, a filament and/or particle and/or fibrous composition may comprise 25% by weight on a dry filament basis and/or dry particle basis and/or dry fibrous composition basis of an anionic surfactant, 15% by weight on a dry filament basis and/or dry particle basis and/or dry fibrous composition basis of a nonionic surfactant, 10% by weight of a chelant on a dry filament basis and/or dry particle basis and/or dry fibrous composition basis, and 5% by weight of a perfume a dry filament basis and/or dry particle basis and/or dry fibrous composition basis so that the total level of active agents present in the filament and/or particle and/or fibrous composition is greater than 50%; namely 55% by weight on a dry filament basis and/or dry particle basis and/or dry fibrous composition basis.

“Fibrous composition product” as used herein means a solid form, for example a rectangular solid, sometimes referred to as a sheet, that comprises one or more active agents, for example a fabric care active agent, a dishwashing active agent, a hard surface active agent, and mixtures thereof. In one example, a fibrous composition product of the present invention comprises one or more surfactants, one or more enzymes (such as in the form of an enzyme prill and/or an enzyme liquid), one or more perfumes and/or one or more suds suppressors.

In one example, one or more active agents, in particle or liquid form, may be deposited onto one or more surfaces of the fibrous compositions of the present invention. For example, enzyme suspensions, perfumes, microcapsule slurries, oils, silicones, surfactant pastes, sometimes referred to herein as minors, may be deposited onto one or more surfaces of the fibrous compositions during making of the fibrous compositions and/or converting of the fibrous compositions. Such application may reside on the surface of the fibrous layer or may substantially imbibe into the fibrous composition.

In another example, a fibrous composition product of the present invention comprises a builder and/or a chelating agent. In another example, a fibrous composition product of the present invention comprises a bleaching agent (such as an encapsulated bleaching agent).

“Different from” or “different” as used herein means, with respect to a material, such as a filament as a whole and/or a filament-forming material within a filament and/or an active agent within a filament, that one material, such as a filament and/or a filament-forming material and/or an active agent, is chemically, physically and/or structurally different from another material, such as a filament and/or a filament-forming material and/or an active agent. For example, a filament-forming material in the form of a filament is different from the same filament-forming material in the form of a fiber. Likewise, a starch polymer is different from a cellulose polymer. However, different molecular weights of the same material, such as different molecular weights of a starch, are not different materials from one another for purposes of the present invention.

“Random mixture of polymers” as used herein means that two or more different filament-forming materials are randomly combined to form a filament. Accordingly, two or more different filament-forming materials that are orderly combined to form a filament, such as a core and sheath bicomponent filament, is not a random mixture of different filament-forming materials for purposes of the present invention.

“Associate,” “Associated,” “Association,” and/or “Associating” as used herein with respect to filaments and/or particle means combining, either in direct contact or in indirect contact, filaments and/or particles such that a fibrous composition is formed. In one example, the associated filaments and/or particles may be bonded together for example by adhesives and/or thermal bonds. In another example, the filaments and/or particles may be associated with one another by being deposited onto the same fibrous composition making belt and/or patterned belt.

“Machine Direction” or “MD” as used herein means the direction parallel to the flow of the fibrous composition through the fibrous composition making machine and/or fibrous composition product manufacturing equipment.

“Cross Machine Direction” or “CD” as used herein means the direction perpendicular to the machine direction in the same plane of the fibrous composition and/or fibrous composition product comprising the fibrous composition.

“Ply” or “Plies” as used herein means an individual fibrous composition optionally to be disposed in a substantially contiguous, face-to-face relationship with other plies, forming a multiple ply fibrous composition. It is also contemplated that a single fibrous composition can effectively form two “plies” or multiple “plies”, for example, by being folded on itself. A ply may comprise layers of filaments, filament/particle blends, and/or particles. In another embodiment, there may be a layer of filaments or particles between plies.

As used herein, the articles “a” and “an” when used herein, for example, “an anionic surfactant” or “a fiber” is understood to mean one or more of the material that is claimed or described.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

The term “oral care composition” as used herein means a product that in the ordinary course of usage is retained in the oral cavity for a time sufficient to contact some or all of the dental surfaces and/or oral tissues for purposes of oral health. In one embodiment, the composition is retained in the oral cavity to deliver an oral care active agent. The oral composition of the present invention may be in various forms including toothpaste, dentifrice, tooth gel, tooth powders, tablets, rinse, sub gingival gel, foam, mousse, chewing gum, lipstick, sponge, floss, prophy paste, petrolatum gel, denture product, nonwoven web, or foam. In one embodiment, the oral composition is in the form of a nonwoven web. In another embodiment, the oral composition is in the form of a dentifrice. The oral composition may also be incorporated onto strips or films for direct application or attachment to oral surfaces or incorporated into floss. The oral care composition may also be a strip that can be directly applied to a surface of the oral cavity. The strip can at least partially dissolve upon contact with moisture or brushing.

The term “orally acceptable carrier” as used herein means a suitable vehicle or ingredient, which can be used to form and/or apply the present compositions to the oral cavity in a safe and effective manner

The term “effective amount” as used herein means an amount of a compound or composition sufficient to induce a positive benefit, an oral health benefit, and/or an amount low enough to avoid serious side effects, i.e., to provide a reasonable benefit to risk ratio, within the sound judgment of a skilled artisan. Depending on the type of oral health benefit and the efficacy of active compound, “effective amount” means at least about 0.0001% of the material, 0.001% of the material, or 0.01 of the material, by weight of the composition.

The term “dentifrice” as used herein means paste, gel, powder, tablets, or liquid formulations, unless otherwise specified, that are used to clean, treat, or contact the surfaces of the oral cavity. Additionally, as disclosed herein, the dentifrice means a nonwoven web that are used to clean the surfaces of the oral cavity. The term “teeth” as used herein refers to natural teeth as well as artificial teeth or dental prosthesis.

### Process for Making an Oral Care Article of Manufacture

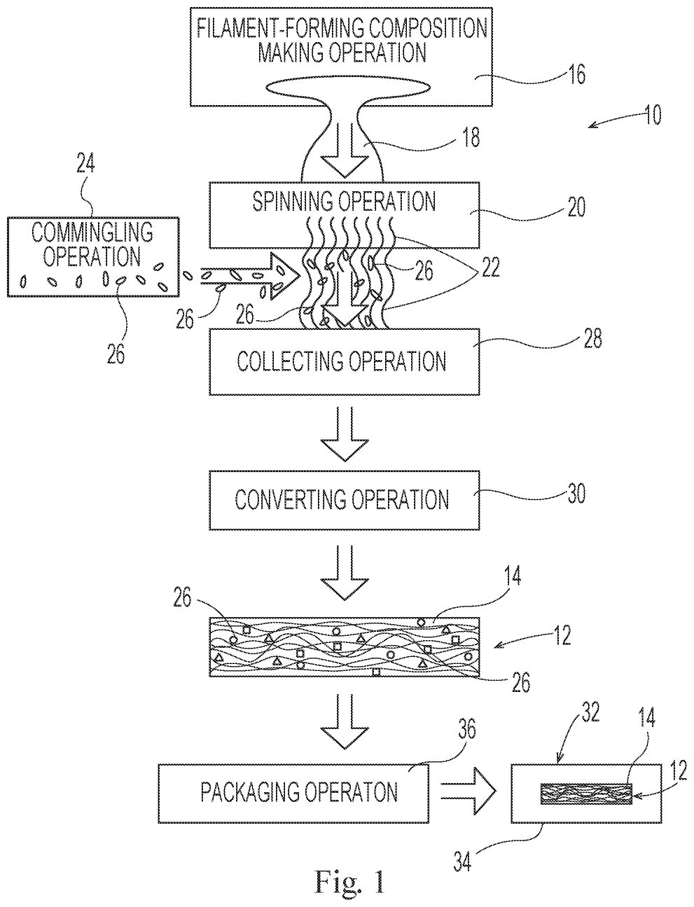

In one example of the present invention, as shown in FIG. 1, a process 10 for making, for example continuously making, an oral care article of manufacture 12 comprising a fibrous composition 14, for example a soluble fibrous composition, comprises at least the following steps: 1) a filament-forming composition making operation 16 comprising one or more steps to make a filament-forming composition 18, which is then delivered, for example via piping, to the next operation; namely, a spinning operation 20; 2) a spinning operation 20 comprising one or more steps for spinning a filament-forming composition, for example the filament-forming composition 18 made in the filament-forming composition making operation 16, to make filaments 22, for example soluble filaments; 3) optionally, a commingling operation 24 comprising one or more steps of commingling, for example coforming, a plurality of solid additives, for example particles 26, with the filaments 22; and 4) a collecting operation 28 comprising one or more collection steps for collecting the filaments 22 and/or commingled filaments 22 and solid additives, for example particles 26, on a collection device, such as a belt and/or rotary drum, to form a fibrous composition 14 comprising filaments 22 and optionally, solid additives, for example particles 26, for example a soluble fibrous composition, wherein the operations (1-4) when present are performed in a continuous manner, one step after the other without any breaks or stoppages or interruptions in the process from making a filament-forming composition 18 to spinning the filament-forming composition 18 into filaments 22 (optionally commingling solid additives, for example particles 26, with the filaments 22) to collecting the filaments 22 (and/or commingled filaments 22 and solid additives, for example particles 26) on a collection device to form a fibrous composition 14 comprising filaments 22 and optionally, solid additives, for example particles 26.

Once the fibrous composition 14 is formed, then the fibrous composition 14 may then be converted into an oral care article of manufacture 12, which may be a consumable, saleable unit, via a converting operation 30, which comprises one or more steps for converting the fibrous composition 14 into an oral care article of manufacture 12, wherein one or more of the converting steps may be continuous with the earlier operations (1-4) of the process 10. Once the fibrous composition 14 has been converted into an oral care article of manufacture 12 comprising the fibrous composition 14, for example one or more, two or more, three or more, four or more, five or more plies of the fibrous composition 14, then the article of manufacture 12 can be packaged into a package 32 comprising an external packaging material 34, such as a packaging film, a cardboard box, and the like, via a packaging operation 36.

In one example, the process for making an oral care article of manufacture and/or process steps such as the spinning operation, the commingling (coforming) operation, the collecting operation, the converting operation, and the packaging operation, according to the present invention is performed at a relative humidity of from about 20% to about 75% and/or from about 30% to about 65% and/or from about 35% to about 60%.

The converting operation 30 may comprise one or more steps for converting (for example slitting and/or stacking and/or calendering and/or treating with optional ingredients (such as adding optional ingredients to the fibrous composition 14, for example to a surface of the fibrous composition 14), such as perfumes, enzymes, bleaches, flavoring agents, effervescent agents, and the like, die-cutting, and printing) the fibrous composition 14 into one or more articles of manufacture 12, for example a consumer product); and 6) optionally, a packaging operation 36 comprising one or more steps for packaging one or more articles of manufacture 12, for example a consumer product, such as a soluble consumer product, into a package 32.

In one example, the converting operation may include die cutting into a desired shape, for example to maximize the number of articles of manufacture produced from a fibrous composition or multiple desired shapes, printing, optional ingredient (minors) additions, rolling up a fibrous composition on a roll as converting line step, including where all this is done in a single process or on a single converting line. For example, the process of the present invention may comprise one or more converting operations and/or steps selected from the group consisting of: slitting, stacking, calendering, treating with optional ingredients, die cutting, printing, packaging, and/or combinations thereof. In one example, one or more or all of these converting operations and/or steps are performed on a single converting line, which may be directly coupled to the fibrous composition making line (for example spinning/commingling/collecting operations) and the filament-forming composition making operation. In one example, as discussed herein, the total process from filament-forming composition operation through the converting operation, and optionally the packaging operation to make an oral care article of manufacture according to the present invention may occur on a single manufacturing line, for example a single, continuous manufacturing line. The converting operation may ultimately yield a consumer useable saleable unit.

In one example, process is such that a fibrous composition, for example a composite structure, formed in the collecting operation is further transformed with converting operations and/or steps selected from the group consisting of: slitting, stacking, calendering, treating with optional ingredients, die cutting, printing, packaging, and/or combinations thereof on a unitary manufacturing line into a consumer use saleable unit.

### a. Filament-Forming Composition Making Operation (16)

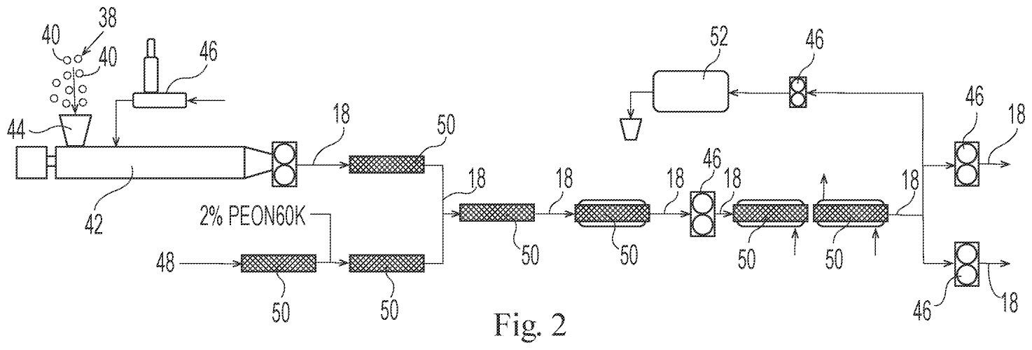

As shown in FIG. 2, in one example, a filament-forming composition 18 is made by providing one or more filament-forming materials 38, for example one or more soluble filament-forming materials, for example one or more hydroxyl polymers, such as polyvinyl alcohol, to which water or another polar solvent are added resulting in an aqueous or polar solvent composition comprising the soluble filament-forming materials and water or polar solvent. The aqueous or polar solvent composition is then processed, for example polymer processed, in an extruder to form a filament-forming composition 18, which is then suitable for delivering to one or more dies for spinning into a plurality of filaments 22 in a spinning operation 20. In one example, the aqueous or polar solvent composition may be processed in a batch tank (not shown).

In one example, the filament-forming material 38 is sufficiently cooked to form a homogeneous aqueous or polar solvent composition of the filament-forming material 38.

In one example, at least about 30% and/or at least about 40% and/or to about 70% and/or to about 60% by weight of water is added to the one or more filament-forming materials 38 during the filament-forming composition making operation 16.

In one example, the filament-forming material 38 may be added at a solids concentration of greater than 40% and/or greater than 50% and/or greater than 60% and/or from about 60% to about 80% and/or from about 60% to about 70%.

In one example, the filament-forming material 38 may be present in the melt composition at a level of greater than 5% and/or greater than 10% and/or greater than 13% and/or less than 50% and/or less than 40% and/or less than 30% and/or less than 25%

In one example, the filament-forming material 38 may be present in the extruder at a level of greater than 10% and/or greater than 20% and/or greater than 30% and/or less than 90% and/or less than 80% and/or less than 70% and/or less than 65%.

In one example, the filament-forming material 38 may be in solid form, for example in a dry solid form 40, such as pellets and/or powder. In one example, the filament-forming material 38 and water and/or another polar solvent utilized to solubilize the filament-forming material 38 are added to an extruder 42 via a hopper 44, for example a twin screw extruder, and heated, processed, and mixed to solubilize the filament-forming material 38. In one example, entrained air within the aqueous solution and/or polar solvent solution comprising the filament-forming material 18 within the extruder 40 is minimized and/or eliminated. The water and/or polar solvent may be added to the extruder 42 via a pump 46.

When the filament-forming material 38 is in solid form, the solid filament-forming material, for example one or more hydroxyl polymers, such as polyvinyl alcohol, is added from a hopper 44, for example in a continuous process, to an extruder 42, for example a single screw extruder or twin screw extruder, for example a twin screw extruder, such as a Coperion ZSK 26 twin screw extruder (Max. Speed 1200 rpm, Max. Torque per Screw Shaft 106 Nm, Diameter of Screws 25.5 mm, Length of Screws 900 mm, # of Barrel Sections 9, Heating and Cooling for each Zone, Flight Depth 4.55 mm, Est. Throughput 20-60 kg/hr). In this example, as shown in

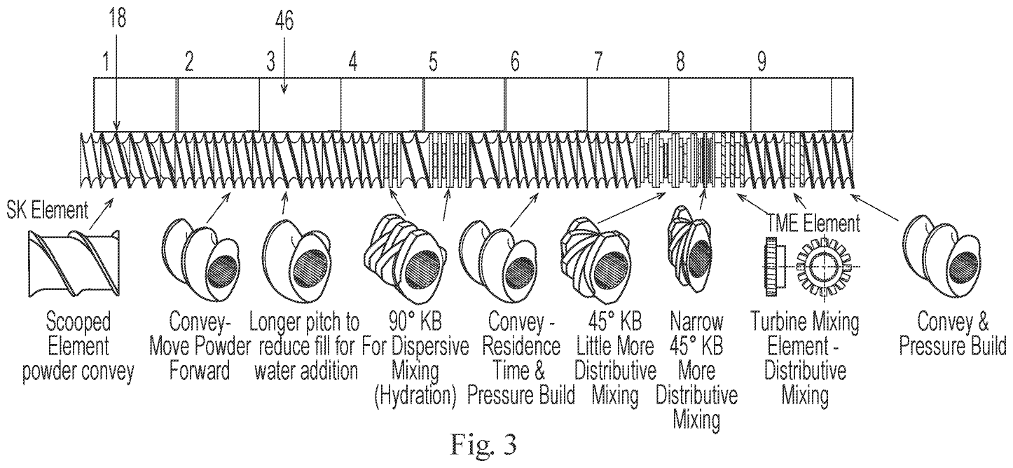

FIG. 3 the addition of the solid filament-forming material to the extruder 42 occurs in Zone 1 of the extruder 42. The purpose of adding the filament-forming material 38 to an extruder 42 is to achieve hydration of the filament-forming material 38 and to solubilize the filament-forming material 38, especially if it is originally in a solid form.

Non-limiting examples of suitable filament-forming materials 38 include polymers, for example polymers selected from the group consisting of: pullulan, hydroxypropylmethyl cellulose, hydroxyethyl cellulose, hydroxypropyl cellulose, methyl cellulose, polyvinyl pyrrolidone, carboxymethyl cellulose, sodium alginate, xanthan gum, tragacanth gum, guar gum, acacia gum, Arabic gum, polyacrylic acid, methylmethacrylate copolymer, carboxyvinyl polymer, dextrin, pectin, chitin, levan, elsinan, collagen, gelatin, zein, gluten, soy protein, casein, polyvinyl alcohol, carboxylated polyvinyl alcohol, sulfonated polyvinyl alcohol, starch, starch derivatives, hemicellulose, hemicellulose derivatives, proteins, chitosan, chitosan derivatives, polyethylene glycol, tetramethylene ether glycol, hydroxymethyl cellulose, polyethylene oxide, and mixtures thereof.

In one example, the filament-forming material 38 is a water-soluble material that produces a soluble filament, for example a water-soluble filament.

In one example, the filament-forming material 38 comprises polyvinyl alcohol.

Water and/or another polar solvent is added via a pump 46, for example in a continuous process, to the extruder 42 containing the filament-forming material 18, to mix with and solubilize the filament-forming material 18 within the extruder 42. The water and/or other polar solvent are added to the extruder 42 in Zone 3.

The extruder 42 may be run such that it exhibits a wet throughput of at least about 5 and/or at least about 10 and/or at least about 15 and/or at least about 20 and/or at least about 40 at least about 80 and/or from about 15 to about 200 and/or from about 80 to about 135 kg/hr, in one example to produce a full filament-forming material flow rate of from about 100 to about 700 kg/hr and/or from about 345 to about 575 kg/hr, a dry throughput of at least about 2 and/or at least about 4 and/or at least about 6 and/or at least about 10 and/or at least about 15 and/or at least about 20 and/or from about 2 to about 120 and/or from about 10 to about 85 and/or from about 20 to about 85 kg/hr and/or from about 50 to about 85 kg/hr, a maximum screw speed of less than about 1600 rpm and/or less than about 1400 rpm and/or less than about 1200 rpm and/or from about about 200 to about 1600 rpm and/or from about 400 to 1400 rpm and/or from about 600 to about 1200 rpm, a % solids (filament-forming material 38) of from about 20 to about 95% and/or from about 30 to about 85% and/or from about 40 to about 70%, an exit pressure of from about 10 to about 80 and/or from about 15 to about 75 and/or from about about 20 to about 65 bar setpoint, the filament-forming composition may exit the extruder at a SME (solids throughput basis) of from about 0.10 to about 0.50 and/or from about 0.12 to about 0.45 and/or from about 0.14 to about 0.35 kW-h/kg, and wherein the extruder subjects the filament-forming composition to a temperature of at least 49° C., with example barrel temperatures of the extruder run as shown in Table 1 below:

TABLE 1

| Zone | 2 | 3 | 4 | 5 | 6 | 7 | 8 | 9 | 8-0 | Pump | Die |
|---|---|---|---|---|---|---|---|---|---|---|---|
| Temp Setpoint (° C.) “A” | 25 | 49 | 66 | 121 | 121 | 135 | 135 | 135 | 135 | 135 | 135 |
| Temp Setpoint (° C.) “B” | 40 | 100 | 150 | 150 | 150 | 150 | 150 | 150 | 150 | 150 | 150 |
| Temp Setpoint (° C.) “C” | 40 | 100 | 150 | 150 | 160 | 160 | 160 | 160 | 160 | 160 | 160 |
| Temp Setpoint (° C.) “D” | 40 | 100 | 150 | 150 | 160 | 170 | 170 | 170 | 170 | 170 | 170 |
| Temp Setpoint (° C.) “E” | 40 | 80 | 80 | 130 | 140 | 140 | 170 | 170 | 170 | 170 | 170 |

In addition to solubilizing the filament-forming material 38 in an extruder 42 to produce a filament-forming composition 18, one or more active agents 48, for example one or more surfactants, such as a surfactant blend, for example a blend of anionic surfactants, may be mixed with the filament-forming composition 18 via one or more static mixers 50, such as SMX mixers.

In one example, the surfactant and/or surfactant blend comprises one or more anionic surfactants selected from the group consisting of: linear alkylbenzene sulfonates (LAS), alkyl sulfates (AS), and mixtures thereof. The surfactants may be blended and or co-neutralized with sodium hydroxide to form a low water containing paste. In addition, other surfactants, such as alyklethoxylate sulfates (AES), cosurfactants, such as amine oxide, linear alcohol ethoxylates, glucamide-based surfactants, and branched versions of the alkyl chain, such as MLAS and HSAS.

In one example, in addition to the one or more surfactants, structurant, such as polyethylene oxide, such as a PEO 100K and/or PEO N60K, and/or polyvinylpyrrolidone, may be mixed with the surfactants to provide phase stability. Optionally, other ingredients may also be mixed with the surfactants, such as salts, for example sodium sulfate.

In one example, the filament-forming composition 18 and thus at least one filament 22 produced from spinning the filament-forming composition 18 comprises one or more active agents 48, in the case of the filament 22 the one or more active agents 48 are present within the filament 22.

In one example, the active agent 48 comprises a surfactant selected from the group consisting of: anionic surfactants, cationic surfactants, nonionic surfactants, zwitterionic surfactants, amphoteric surfactants, and mixtures thereof.

In one example, the one or more active agents 48 is selected from the group consisting of: fabric care active agents, dishwashing active agents, carpet care active agents, surface care active agents, air care active agents, oral care active agents (for example teeth cleaning agents, teeth whitening agents, tooth care agents, periodontal gum care agents, mouthwash agents, denture cleaning agents, tongue cleaning agents, breath freshening agents, fluoride agents, mouth rinse agents, anti-cavity agents, flavoring agents), hair care active agents (shampoos and/or conditioners), keratinaceous tissue care agents, toilet bowl cleaning agents, skin care active agents, and mixtures thereof.

In one example, at least one of the active agents 48 comprises one or more effervescent agents.

In one example, one or more hueing agents, colorants, and/or dyes are added to the filament-forming composition during the filament-forming composition making operation.

### Filament-forming Material

The filament-forming material is any suitable material, such as a polymer or monomers capable of producing a polymer that exhibits properties suitable for making a fibrous element, such as by a spinning process.

In one example, the filament-forming material may comprise a polar solvent-soluble material, such as an alcohol-soluble material and/or a water-soluble material.

In another example, the filament-forming material may comprise a non-polar solvent-soluble material.

In still another example, the filament forming material may comprise a polar solvent-soluble material and be free (less than 5% and/or less than 3% and/or less than 1% and/or 0% by weight on a dry fibrous element basis and/or dry soluble fibrous composition basis) of non-polar solvent-soluble materials.

In yet another example, the filament-forming material may be a film-forming material. In still yet another example, the filament-forming material may be synthetic or of natural origin and it may be chemically, enzymatically, and/or physically modified.

In even another example of the present invention, the filament-forming material may comprise a polymer selected from the group consisting of: polymers derived from acrylic monomers such as the ethylenically unsaturated carboxylic monomers and ethylenically unsaturated monomers, polyvinyl alcohol, polyacrylates, polymethacrylates, copolymers of acrylic acid and methyl acrylate, polyvinylpyrrolidones, polyalkylene oxides, starch and starch derivatives, pullulan, gelatin, hydroxypropylmethylcelluloses, methyl celluloses, and carboxymethycelluloses.

In still another example, the filament-forming material may comprises a polymer selected from the group consisting of: polyvinyl alcohol, polyvinyl alcohol derivatives, starch, starch derivatives, cellulose derivatives, hemicellulose, hemicellulose derivatives, proteins, sodium alginate, hydroxypropyl methylcellulose, chitosan, chitosan derivatives, polyethylene glycol, tetramethylene ether glycol, polyvinyl pyrrolidone, hydroxymethyl cellulose, hydroxyethyl cellulose, methyl cellulose, and mixtures thereof.

In another example, the filament-forming material comprises a polymer is selected from the group consisting of: pullulan, hydroxypropylmethyl cellulose, hydroxyethyl cellulose, hydroxypropyl cellulose, methyl cellulose, polyvinyl pyrrolidone, carboxymethyl cellulose, methyl cellulose, sodium alginate, xanthan gum, tragacanth gum, guar gum, acacia gum, Arabic gum, polyacrylic acid, methylmethacrylate copolymer, carboxyvinyl polymer, dextrin, pectin, chitin, levan, elsinan, collagen, gelatin, zein, gluten, soy protein, casein, polyvinyl alcohol, starch, starch derivatives, hemicellulose, hemicellulose derivatives, proteins, chitosan, chitosan derivatives, polyethylene glycol, tetramethylene ether glycol, hydroxymethyl cellulose, and mixtures thereof.

### Polar Solvent-soluble Materials

Non-limiting examples of polar solvent-soluble materials include polar solvent-soluble polymers. The polar solvent-soluble polymers may be synthetic or natural original and may be chemically and/or physically modified. In one example, the polar solvent-soluble polymers exhibit a weight average molecular weight of at least 10,000 g/mol and/or at least 20,000 g/mol and/or at least 40,000 g/mol and/or at least 80,000 g/mol and/or at least 100,000 g/mol and/or at least 1,000,000 g/mol and/or at least 3,000,000 g/mol and/or at least 10,000,000 g/mol and/or at least 20,000,000 g/mol and/or to about 40,000,000 g/mol and/or to about 30,000,000 g/mol.

In one example, the polar solvent-soluble polymers are selected from the group consisting of: alcohol-soluble polymers, water-soluble polymers and mixtures thereof. Non-limiting examples of water-soluble polymers include water-soluble hydroxyl polymers, water-soluble thermoplastic polymers, water-soluble biodegradable polymers, water-soluble non-biodegradable polymers and mixtures thereof. In one example, the water-soluble polymer comprises polyvinyl alcohol. In another example, the water-soluble polymer comprises starch. In yet another example, the water-soluble polymer comprises polyvinyl alcohol and starch.

a. Water-soluble Hydroxyl Polymers—Non-limiting examples of water-soluble hydroxyl polymers in accordance with the present invention include polyols, such as polyvinyl alcohol, polyvinyl alcohol derivatives, polyvinyl alcohol copolymers, starch, starch derivatives, starch copolymers, chitosan, chitosan derivatives, chitosan copolymers, cellulose derivatives such as cellulose ether and ester derivatives, cellulose copolymers, hemicellulose, hemicellulose derivatives, hemicellulose copolymers, gums, arabinans, galactans, proteins and various other polysaccharides and mixtures thereof.

In one example, a water-soluble hydroxyl polymer of the present invention comprises a polysaccharide. “Polysaccharides” as used herein means natural polysaccharides and polysaccharide derivatives and/or modified polysaccharides. Suitable water-soluble polysaccharides include, but are not limited to, starches, starch derivatives, chitosan, chitosan derivatives, cellulose derivatives, hemicellulose, hemicellulose derivatives, gums, arabinans, galactans and mixtures thereof. The water-soluble polysaccharide may exhibit a weight average molecular weight of from about 10,000 to about 40,000,000 g/mol and/or greater than 100,000 g/mol and/or greater than 1,000,000 g/mol and/or greater than 3,000,000 g/mol and/or greater than 3,000,000 to about 40,000,000 g/mol.

The water-soluble polysaccharides may comprise non-cellulose and/or non-cellulose derivative and/or non-cellulose copolymer water-soluble polysaccharides. Such non-cellulose water-soluble polysaccharides may be selected from the group consisting of: starches, starch derivatives, chitosan, chitosan derivatives, hemicellulose, hemicellulose derivatives, gums, arabinans, galactans and mixtures thereof.

In another example, a water-soluble hydroxyl polymer of the present invention comprises a non-thermoplastic polymer.

The water-soluble hydroxyl polymer may have a weight average molecular weight of from about 10,000 g/mol to about 40,000,000 g/mol and/or greater than 100,000 g/mol and/or greater than 1,000,000 g/mol and/or greater than 3,000,000 g/mol and/or greater than 3,000,000 g/mol to about 40,000,000 g/mol. Higher and lower molecular weight water-soluble hydroxyl polymers may be used in combination with hydroxyl polymers having a certain desired weight average molecular weight.

Well known modifications of water-soluble hydroxyl polymers, such as natural starches, include chemical modifications and/or enzymatic modifications. For example, natural starch can be acid-thinned, hydroxy-ethylated, hydroxy-propylated, and/or oxidized. In addition, the water-soluble hydroxyl polymer may comprise dent corn starch.

Naturally occurring starch is generally a mixture of linear amylose and branched amylopectin polymer of D-glucose units. The amylose is a substantially linear polymer of D-glucose units joined by (1,4)-α-D links. The amylopectin is a highly branched polymer of D-glucose units joined by (1,4)-α-D links and (1,6)-α-D links at the branch points. Naturally occurring starch typically contains relatively high levels of amylopectin, for example, corn starch (64-80% amylopectin), waxy maize (93-100% amylopectin), rice (83-84% amylopectin), potato (about 78% amylopectin), and wheat (73-83% amylopectin). Though all starches are potentially useful herein, the present invention is most commonly practiced with high amylopectin natural starches derived from agricultural sources, which offer the advantages of being abundant in supply, easily replenishable and inexpensive.

As used herein, “starch” includes any naturally occurring unmodified starches, modified starches, synthetic starches and mixtures thereof, as well as mixtures of the amylose or amylopectin fractions; the starch may be modified by physical, chemical, or biological processes, or combinations thereof. The choice of unmodified or modified starch for the present invention may depend on the end product desired. In one embodiment of the present invention, the starch or starch mixture useful in the present invention has an amylopectin content from about 20% to about 100%, more typically from about 40% to about 90%, even more typically from about 60% to about 85% by weight of the starch or mixtures thereof.

Suitable naturally occurring starches can include, but are not limited to, corn starch, potato starch, sweet potato starch, wheat starch, sago palm starch, tapioca starch, rice starch, soybean starch, arrow root starch, amioca starch, bracken starch, lotus starch, waxy maize starch, and high amylose corn starch. Naturally occurring starches particularly, corn starch and wheat starch, are the preferred starch polymers due to their economy and availability.

Polyvinyl alcohols herein can be grafted with other monomers to modify its properties. A wide range of monomers has been successfully grafted to polyvinyl alcohol. Non-limiting examples of such monomers include vinyl acetate, styrene, acrylamide, acrylic acid, 2-hydroxyethyl methacrylate, acrylonitrile, 1,3-butadiene, methyl methacrylate, methacrylic acid, maleic acid, itaconic acid, sodium vinylsulfonate, sodium allylsulfonate, sodium methylallyl sulfonate, sodium phenylallylether sulfonate, sodium phenylmethallylether sulfonate, 2-acrylamido-methyl propane sulfonic acid (AMPs), vinylidene chloride, vinyl chloride, vinyl amine and a variety of acrylate esters.

In one example, the water-soluble hydroxyl polymer is selected from the group consisting of: polyvinyl alcohols, hydroxymethylcelluloses, hydroxyethylcelluloses, hydroxypropylmethylcelluloses methyl cellulose, and mixtures thereof. A non-limiting example of a suitable polyvinyl alcohol includes those commercially available from Sekisui Specialty Chemicals America, LLC (Dallas, Tex.) under the CELVOL® trade name A non-limiting example of a suitable hydroxypropylmethylcellulose includes those commercially available from the Dow Chemical Company (Midland, Mich.) under the METHOCEL® trade name including combinations with above mentioned hydroxypropylmethylcelluloses.

b. Water-soluble Thermoplastic Polymers—Non-limiting examples of suitable water-soluble thermoplastic polymers include thermoplastic starch and/or starch derivatives, polylactic acid, polyhydroxyalkanoate, polycaprolactone, polyesteramides and certain polyesters, and mixtures thereof.

The water-soluble thermoplastic polymers of the present invention may be hydrophilic or hydrophobic. The water-soluble thermoplastic polymers may be surface treated and/or internally treated to change the inherent hydrophilic or hydrophobic properties of the thermoplastic polymer.

The water-soluble thermoplastic polymers may comprise biodegradable polymers.

Any suitable weight average molecular weight for the thermoplastic polymers may be used. For example, the weight average molecular weight for a thermoplastic polymer in accordance with the present invention is greater than about 10,000 g/mol and/or greater than about 40,000 g/mol and/or greater than about 50,000 g/mol and/or less than about 500,000 g/mol and/or less than about 400,000 g/mol and/or less than about 200,000 g/mol.

### Non-polar Solvent-soluble Materials

Non-limiting examples of non-polar solvent-soluble materials include non-polar solvent-soluble polymers. Non-limiting examples of suitable non-polar solvent-soluble materials include cellulose, chitin, chitin derivatives, polyolefins, polyesters, copolymers thereof, and mixtures thereof. Non-limiting examples of polyolefins include polypropylene, polyethylene and mixtures thereof. A non-limiting example of a polyester includes polyethylene terephthalate.

The non-polar solvent-soluble materials may comprise a non-biodegradable polymer such as polypropylene, polyethylene and certain polyesters.

Any suitable weight average molecular weight for the thermoplastic polymers may be used. For example, the weight average molecular weight for a thermoplastic polymer in accordance with the present invention is greater than about 10,000 g/mol and/or greater than about 40,000 g/mol and/or greater than about 50,000 g/mol and/or less than about 500,000 g/mol and/or less than about 400,000 g/mol and/or less than about 200,000 g/mol.

### Active Agents

Active agents are a class of fiber adjunct that are designed and intended to provide a benefit to something other than the fibrous element and/or particle and/or soluble fibrous composition itself, such as providing a benefit to an environment external to the fibrous element and/or particle and/or soluble fibrous composition. Active agents may be any suitable fiber adjunct that produces an intended effect under intended use conditions of the fibrous element. For example, the active agent may be selected from the group consisting of: personal cleansing and/or conditioning agents such as hair care agents such as shampoo agents and/or hair colorant agents, hair conditioning agents, skin care agents, sunscreen agents, and skin conditioning agents; laundry care and/or conditioning agents such as fabric care agents, fabric conditioning agents, fabric softening agents, fabric anti-wrinkling agents, fabric care anti-static agents, fabric care stain removal agents, soil release agents, dispersing agents, suds suppressing agents, suds boosting agents, anti-foam agents, and fabric refreshing agents; liquid and/or powder dishwashing agents (for hand dishwashing and/or automatic dishwashing machine applications), hard surface care agents, and/or conditioning agents and/or polishing agents; other cleaning and/or conditioning agents such as antimicrobial agents, antibacterial agents, antifungal agents, fabric hueing agents, perfume, bleaching agents (such as oxygen bleaching agents, hydrogen peroxide, percarbonate bleaching agents, perborate bleaching agents, chlorine bleaching agents), bleach activating agents, chelating agents, builders, lotions, brightening agents, air care agents, carpet care agents, dye transfer-inhibiting agents, clay soil removing agents, anti-redeposition agents, polymeric soil release agents, polymeric dispersing agents, alkoxylated polyamine polymers, alkoxylated polycarboxylate polymers, amphilic graft copolymers, dissolution aids, buffering systems, water-softening agents, water-hardening agents, pH adjusting agents, enzymes, flocculating agents, effervescent agents, preservatives, cosmetic agents, make-up removal agents, lathering agents, deposition aid agents, coacervate-forming agents, clays, thickening agents, latexes, silicas, drying agents, odor control agents, antiperspirant agents, cooling agents, warming agents, absorbent gel agents, anti-inflammatory agents, dyes, pigments, acids, and bases; liquid treatment active agents; agricultural active agents; industrial active agents; ingestible active agents such as medicinal agents, teeth whitening agents, tooth care agents, mouthwash agents, periodontal gum care agents, edible agents, dietary agents, vitamins, minerals; water-treatment agents such as water clarifying and/or water disinfecting agents, and mixtures thereof.

Non-limiting examples of suitable cosmetic agents, skin care agents, skin conditioning agents, hair care agents, and hair conditioning agents are described in CTFA Cosmetic Ingredient Handbook, Second Edition, The Cosmetic, Toiletries, and Fragrance Association, Inc. 1988, 1992.

One or more classes of chemicals may be useful for one or more of the active agents listed above. For example, surfactants may be used for any number of the active agents described above. Likewise, bleaching agents may be used for fabric care, hard surface cleaning, dishwashing and even teeth whitening. Therefore, one of ordinary skill in the art will appreciate that the active agents will be selected based upon the desired intended use of the fibrous element and/or particle and/or soluble fibrous composition made therefrom.

For example, if the fibrous element and/or particle and/or soluble fibrous composition made therefrom is to be used for hair care and/or conditioning then one or more suitable surfactants, such as a lathering surfactant could be selected to provide the desired benefit to a consumer when exposed to conditions of intended use of the fibrous element and/or particle and/or soluble fibrous composition incorporating the fibrous element and/or particle.

In one example, if the fibrous element and/or particle and/or soluble fibrous composition made therefrom is designed or intended to be used for laundering clothes in a laundry operation, then one or more suitable surfactants and/or enzymes and/or builders and/or perfumes and/or suds suppressors and/or bleaching agents could be selected to provide the desired benefit to a consumer when exposed to conditions of intended use of the fibrous element and/or particle and/or soluble fibrous composition incorporating the fibrous element and/or particle. In another example, if the fibrous element and/or particle and/or soluble fibrous composition made therefrom is designed to be used for laundering clothes in a laundry operation and/or cleaning dishes in a dishwashing operation, then the fibrous element and/or particle and/or soluble fibrous composition may comprise a laundry detergent composition or dishwashing detergent composition or active agents used in such compositions. In still another example, if the fibrous element and/or particle and/or soluble fibrous composition made therefrom is designed to be used for cleaning and/or sanitizing a toilet bowl, then the fibrous element and/or particle and/or soluble fibrous composition made therefrom may comprise a toilet bowl cleaning composition and/or effervescent composition and/or active agents used in such compositions.

In one example, the active agent is selected from the group consisting of: surfactants, bleaching agents, enzymes, suds suppressors, suds boosting agents, fabric softening agents, denture cleaning agents, hair cleaning agents, hair care agents, personal health care agents, hueing agents, and mixtures thereof.

In one example, at least one of the active agents is selected from the group consisting of: skin benefit agents, medicinal agents, lotions, fabric care agents, dishwashing agents, carpet care agents, surface care agents, hair care agents, air care agents, and mixtures thereof.

The active agent can comprise one or more oral care active agents. The one or more oral care active agents can comprise an abrasive, a fluoride ion source, a metal ion source, a calcium ion source, one or more oral care surfactants, a polyphosphate source, an aesthetic agent, a chelant, a whitening agent, a bioactive material, and/or combinations thereof.

The oral care actives can be present in the fibrous composition, the nonfibrous composition, or combinations thereof. There can be different or the same oral care active agents in the fibrous composition than in the nonfibrous composition. There can be a first fibrous composition comprising a particular combination of oral care active agents and a second fibrous composition comprising a different combination of oral care active agents.

The abrasive can be a calcium-containing abrasive, a silica abrasive, a carbonate abrasive, a phosphate abrasive, an alumina abrasive, other suitable abrasives, and/or combinations thereof. Some abrasives may fit into several descriptive categories, such as for example calcium carbonate, which is both a calcium-containing abrasive and a carbonate abrasive.

The calcium-containing abrasive can comprise calcium carbonate, dicalcium phosphate, tricalcium phosphate, calcium orthophosphate, calcium metaphosphate, calcium polyphosphate, calcium hydroxyapatite, and combinations thereof.

The calcium-containing abrasive can comprise calcium carbonate. The calcium-containing abrasive can be selected from the group consisting of fine ground natural chalk, ground calcium carbonate, precipitated calcium carbonate, and combinations thereof.

The carbonate abrasive can comprise sodium carbonate, sodium bicarbonate, calcium carbonate, strontium carbonate, and/or combinations thereof.

The phosphate abrasive cancomprise calcium phosphate, sodium hexametaphosphate, dicalcium phosphate, tricalcium phosphate, calcium orthophosphate, calcium metaphosphate, calcium polyphosphate, a polyphosphate, a pyrophosphate, and/or combinations thereof.

The silica abrasive can comprise fused silica, fumed silica, precipitated silica, hydrated silica, and/or combinations thereof.

The alumina abrasive can comprise polycrystalline alumina, calcined alumina, fused alumina, levigated alumina, hydrated alumina, and/or combinations thereof.

Other suitable abrasives include diatomaceous earth, barium sulfate, wollastonite, perlite, polymethylmethacrylate particles, tospearl, and combinations thereof.

The abrasive can clog the spinning die, thus, the abrasive can be added to the nonfibrous composition.

The fluoride ion source can comprise examples of suitable fluoride ion-yielding materials are disclosed in U.S. Pat. Nos. 3,535,421, and 3,678,154. The fluoride ion source can comprise stannous fluoride, sodium fluoride, potassium fluoride, amine fluoride, sodium monofluorophosphate, zinc fluoride, and/or combinations thereof.

The fluoride ion source and the metal ion source can be the same compound, such as for example, stannous fluoride, which can generate tin ions and fluoride ions. Additionally, the fluoride ion source and the tin ion source can be separate compounds, such as when the metal ion source is stannous chloride and the fluoride ion source is sodium monofluorophosphate or sodium fluoride.

Suitable metal ion sources include stannous ion sources, zinc ion sources, copper ion sources, silver ion sources, magnesium ion sources, iron ion sources, sodium ion sources, and manganese (Mn) ion sources, and/or combinations thereof. The metal ion source can be a soluble or a sparingly soluble compound of stannous, zinc, or copper with inorganic or organic counter ions. Examples include the fluoride, chloride, chlorofluoride, acetate, hexafluorozirconate, sulfate, tartrate, gluconate, citrate, malate, glycinate, pyrophosphate, metaphosphate, oxalate, phosphate, carbonate salts and oxides of stannous, zinc, and copper.

Stannous, zinc and copper ions are derived from the metal ion source(s) can be found in the multi-phase oral care composition an effective amount to provide an oral care benefit or other benefits. Stannous, zinc and copper ions have been found to help in the reduction of gingivitis, plaque, sensitivity, and improved breath benefits.

Other metal ion sources can include minerals and/or calcium containing compounds, which can lead to remineralization, such as, for example, sodium iodide, potassium iodide, calcium chloride, calcium lactate, calcium phosphate, hydroxyapatite, fluoroapatite, amorphous calcium phosphate, crystalline calcium phosphate, sodium bicarbonate, sodium carbonate, calcium carbonate, oxalic acid, dipotassium oxalate, monosodium monopotassium oxalate, casein phosphopeptides, and/or casein phosphopeptide coated hydroxy apatite.

The metal ion source may comprise a metal salt suitable for generating metal ions in the oral cavity. Suitable metal salts include salts of silver (Ag), magnesium (Mg), iron (Fe), sodium (Na), and manganese (Mn) salts, or combinations thereof. Preferred salts include, without limitation, gluconates, chlorates, citrates, chlorides, fluorides, and nitrates, or combinations thereof.

The oral care article can comprise one or more surfactants. The fibrous composition can comprise one or more surfactants. The nonfibrous composition can comprise one or more surfactants. The one or more surfactants may be selected from anionic, nonionic, amphoteric, zwitterionic, cationic surfactants, or combinations thereof, as described herein. A polyphosphate source can comprise one or more polyphosphate molecules.

Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. The polyphosphate source can also include alkali earth metal polyphosphate salts, and specifically calcium polyphosphate salts, such as calcium pyrophosphate, due to the ability to separate calcium ions from other reactive components, such as fluoride ion sources.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

Polyphosphates can degrade under the conditions required to spin a filament from the filament forming composition and/or clog the spinning die, thus, the polyphosphate can be added to the nonfibrous composition.

The one or more aesthetic agents can be selected from the group consisting of flavors, colorants, sensates, sweeteners, salivation agents, and combinations thereof.

Non-limiting examples of flavors that can be used in the present invention can include natural flavoring agents, artificial flavoring agents, artificial extracts, natural extracts and combination thereof. Non-limiting examples of flavors can include vanilla, honey, lemon, lemon honey, cherry vanilla, peach, honey ginger, chamomile, cherry, cherry cream, mint, vanilla mint, dark berry, black berry, raspberry, peppermint, spearmint, honey peach, acai berry, cranberry, honey cranberry, tropical fruit, dragon fruit, wolf berry, red stem mint, pomegranate, black current, strawberry, lemon, lime, peach ginger, orange, orange cream, cream sickle, apricot, anethole, ginger, jack fruit, star fruit, blueberry, fruit punch, lemon grass, chamomile lemon grass, lavender, banana, strawberry banana, grape, blue raspberry, lemon lime, coffee, espresso, cappuccino, honey, wintergreen mint, bubble gum, tart honey lemon, sour lemon, green apple, boysenberry, rhubarb, strawberry rhubarb, persimmon, green tea, black tea, red tea, white tea, honey lime, cherry lime, apple, tangerine, grapefruit, kiwi, pear, vanillin, ethyl vanillin, maltol, ethyl-maltol, pumpkin, carrot cake, white chocolate raspberry, chocolate, white chocolate, milk chocolate, dark chocolate, chocolate marshmallow, apple pie, cinnamon, hazelnut, almond, cream, crème brülée, caramel, caramel nut, butter, butter toffee, caramel toffee, aloe vera, whiskey, rum, cocoa, licorice, pineapple, guava, melon, watermelon, elder berry, mouth cooler, raspberries and cream, peach mango, tropical, cool berry, lemon ice, nectar, spicy nectar, tropical mango, apple butter, peanut butter, tangerine, tangerine lime, marshmallow, cotton candy, apple cider, orange chocolate, adipic acid, citral, denatonium benzoate, ethyl acetate, ethyl lactate, ethyl maltol, ethylcellulose, fumaric acid, leucine, malic acid, menthol, methionine, monosodium glutamate, sodium acetate, sodium lactate, tartaric acid, thymol, and combinations thereof.

Flavors can be protected in an encapsulate or as a flavor crystal. The encapsulated flavor can have a controlled or delayed release once the encapsulated flavor reaches the oral cavity. The encapsulate can comprise a shell and a core. The flavor can be in the core of the encapsulate. The flavor can be encapsulated by any suitable means, such as spray drying or extrusion. Encapsulated flavors can be added to the surface of the fibrous composition, formed within the fibrous composition, or included in the nonfibrous composition.

Flavors can degrade under the conditions required to spin a filament from the filament forming composition, the flavor can be added to the nonfibrous composition.

Non-limiting examples of cooling sensates can include WS-23 (2-Isopropyl-N,2,3-trimethylbutyramide), WS-3 (N-Ethyl-p-menthane-3-carboxamide), WS-30 (1-glyceryl-p-mentane-3-carboxylate), WS-4 (ethyleneglycol-p-methane-3-carboxylate), WS-14 (N-t-butyl-p-menthane-3-carboxamide), WS-12 (N-(4-ethoxyphenyl)-p-menthane-3-carboxamide), WS-5 (Ethyl-3-(p-menthane-3-carboxamido)acetate, Menthone glycerol ketal (sold as Frescolat® MGA by Haarmann & Reimer), (−)-Menthyl lactate (sold as Frescolat® ML by Haarmann & Reimer), (−)-Menthoxypropane-1,2-diol(sold as Coolant Agent 10 by Takasago International), 3-(1-menthoxy)propane-1,2-diol, 3-(1-Menthoxy)-2-methylpropane-1,2-diol, (−)-Isopulegol is sold under the name “Coolact P®” by Takasago International., cis & trans p-Menthane-3,8-diols(PMD38)—Takasago International, Questice® (menthyl pyrrolidone carboxylate), (1R,3R,4S)-3-menthyl-3,6-dioxaheptanoate—Firmenich, (1R,2S,5R)-3-menthyl methoxyacetate—Firmenich, (1R,2S,5R)-3-menthyl 3,6,9-trioxadecanoate—Firmenich, (1R,2S,5R)-menthyl 11-hydroxy-3,6,9-trioxaundecanoate—Firmenich, (1R,2S,5R)-3-menthyl (2-hydroxyethoxy)acetate—Firmenich, Cubebol—Firmenich, Icilin also known as AG-3-5, chemical name 1-[2-hydroxyphenyl]-4-[2-nitrophenyl-]-1,2,3,6-tetrahydropyrimidine-2-one), 4-methyl-3-(1-pyrrolidinyl)-2[5H]-furanone, Frescolat ML—menthyl lactate, Frescolat MGA—menthone glycerin acetal, Peppermint oil, Givaudan 180, L-Monomenthyl succinate, L-monomenthyl glutarate, 3-1-menthoxypropane-1,2-diol—(Coolact 10), 2-1-menthoxyethanol (Cooltact 5), TK10 Coolact (3-1-Menthoxy propane-1,2-diol), Evercool 180 (N-p-benzeneacetonitrile-menthane carboxamide), and combinations thereof. Cooling sensates can be present from about 0.005% to about 10%, by weight of the oral care composition, from about 0.05% to about 7%, by weight of the oral care composition, or from about 0.01% to about 5%, by weight of the oral care composition.

Non-limiting examples of warming sensates can include TK 1000, TK 1 MM, Heatenol—Sensient Flavors, Optaheat—Symrise Flavors, Cinnamon, Polyethylene glycol, Capsicum, Capsaicin, Curry, FSI Flavors, Isobutavan, Ethanol, Glycerin, Nonivamide 60162807, Hotact VEE, Hotact 1 MM, piperine, optaheat 295 832, optaheat 204 656, optaheat 200 349, and combinations thereof. Warming sensates can be present from about 0.005% to about 60%, by weight on a dry filament basis, from about 0.05% to about 50%, by weight on a dry filament basis, or from about 0.01% to about 40%, by weight on a dry filament basis. Warming sensates can be present from about 0.005% to about 10%, by weight of the oral care composition, from about 0.05% to about 7%, by weight of the oral care composition, or from about 0.01% to about 5%, by weight of the oral care composition.

Non-limiting examples of tingling sensates can include sichuan pepper, hydroxy alpha sanshool, citric acid, Jambu extracts, spilanthol, and combinations thereof.

The term “chelant”, as used herein means a bi- or multidentate ligand having at least two groups capable of binding to metal ions and preferably other divalent or polyvalent metal ions and which, at least as part of a chelant mixture, is capable of solubilizing tin ions or other optional metal ions within the oral care composition. Groups capable of binding to metal ions include carboxyl, hydroxl, and amine groups.

Suitable chelants herein include C₂-C₆ dicarboxylic and tricarboxylic acids, such as succinic acid, malic acid, tartaric acid and citric acid; C₃-C₆ monocarboxylic acids substituted with hydroxyl, such as gluconic acid; picolinic acid; amino acids such as glycine; salts thereof and mixtures thereof. The chelants can also be a polymer or copolymer in which the chelating ligands are on the same or adjacent monomer

The whitening agent can be a compound suitable for whitening at least one tooth in the oral cavity. The whitening agent may include peroxides, metal chlorites, perborates, percarbonates, peroxyacids, persulfates, and combinations thereof. Suitable peroxides include solid peroxides, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

Whitening agents can be reactive with other components of oral care compositions, thus, can be separated from other components using the assembly design described herein. Additionally, whitening agents can degrade under the conditions required to spin filaments from the filament forming composition, thus, the whitening agent can be added to the nonfibrous composition. Suitable bioactive materials include bioactive glasses, Novamin™, Recaldent™, hydroxyapatite, amino acids, such as, for example, arginine, citrulline, glycine, lysine, or histidine, or combinations thereof. Other suitable bioactive materials include any calcium phosphate compound. Other suitable bioactive materials include compounds comprising a calcium source and a phosphate source.

Bioactive glasses are comprising calcium and/or phosphate which can be present in a proportion that is similar to hydroxyapatite. These glasses can bond to the tissue and are biocompatible. Bioactive glasses can include a phosphopeptide, a calcium source, phosphate source, a silica source, a sodium source, and/or combinations thereof.

The filament-forming composition 18 may then be mixed via static mixers 50, such as SMX mixers, jacketed or unjacketed, and/or pumped via piping and/or pumps 46, such as a booster pump, to a spinning operation 20. The filament-forming composition 18 produced from the filament-forming composition making operation 16 may be delivered to one or more dies and/or one or more beams of dies via one or more pumps 46. Before being delivered to the spinning operation 20, the rheology of the filament-forming composition 18 may be measured, offline or online, for example with an online rheometer 52, to ensure that the filament-forming composition's 18 rheology is suitable for spinning into filaments 22 via the spinning operation 20.

The formation and/or attenuation of a filament requires a delicate balance of forces to be successful. First, the filament-forming composition 18 must form a stable filament 20 as it exits the die. When the viscosity of the filament-forming composition 18 is too high, full attenuation cannot be achieved. When the viscosity of the filament-forming composition 18 is too low, the filament 20 will break under the attenuation forces. Additionally, after the filament 20 has been attenuated down to about 20 um diameter, stabilization ensues. The stabilization process can be achieved in a number of ways, most notably drying and/or crystallization. The rheological properties of the filament 20 as it transitions from a liquid (filament-forming composition 18) to a solid (filament 20) are of paramount importance in successful filament spinning In one example, the filament-forming composition 18 of the present invention exhibits a Capillary Number of greater than 1 and/or greater than 2 and/or greater than 3 and/or greater than 4 and/or greater than 5. In a fibrous element spinning process, the fibrous elements need to have initial stability as they leave the spinning die. In one example, the filament-forming composition 18 exhibits a Capillary Number of from at least about 1 to about 50 and/or at least about 3 to about 50 and/or at least about 5 to about 30 such that the filament-forming composition 18 can be effectively polymer processed (spun) into a filament 22.

The Capillary Number is a dimensionless number used to characterize the likelihood of this droplet breakup. A larger Capillary Number indicates greater fluid stability upon exiting the die. The Capillary Number is defined as follows:

$$Ca = \frac{V * η}{σ}$$

V is the fluid velocity at the die exit (units of Length per Time),
η is the fluid viscosity at the conditions of the die (units of Mass per Length*Time),
σ is the surface tension of the fluid (units of mass per Time²). When velocity, viscosity, and surface tension are expressed in a set of consistent units, the resulting Capillary Number will have no units of its own; the individual units will cancel out.

The Capillary Number is defined for the conditions at the exit of the die. The fluid velocity is the average velocity of the fluid passing through the die opening. The average velocity is defined as follows:

$$V = \frac{{Vol}^{′}}{Area}$$

Vol′=volumetric flowrate (units of Length³ per Time),
Area=cross-sectional area of the die exit (units of Length²).

When the die opening is a circular hole, then the fluid velocity can be defined as

$$V = \frac{{Vol}^{′}}{π * {R}^{2}}$$

R is the radius of the circular hole (units of length).

The shear viscosity of the filament-forming composition 18 may be in the range of from about 0.1 Pa-s to about 50 Pa-s and/or from about 0.3 Pa-s to about 40 Pa-s and/or from about 0.5 Pa-s to about 35 Pa-s at 3000 s⁻¹ at the operating temperature range of the spinning operation 20. The extensional viscosity of the filament-forming composition 18 may be in the range of from about 50 Pa-s to about 200 Pa-s and/or grom about 60 Pa-s to about 180 Pa-s and/or from about 70 Pa-s to about 150 Pa-s and/or from about 75 Pa-s to about 125 Pa-s and/or from about 75 Pa-s to about 100 Pa-s at a strain rate of 700 s⁻¹ as measured by an e-VROC instrument or equivalent from RheoSense of San Ramon, Calif. The Pressure P23/P14 ratio on SSEVR should be greater than 0.8 and/or greater than 0.9 and/or greater than 1.

In one example, the filament-forming composition may comprise at least 20% and/or at least 30% and/or at least 40% and/or at least 45% and/or at least 50% to about 90% and/or to about 85% and/or to about 80% and/or to about 75% by weight of one or more filament-forming materials, one or more active agents, and mixtures thereof. The filament-forming composition may comprise from about 10% to about 80% by weight of a polar solvent, such as water.

In one example, non-volatile components of the filament-forming composition may comprise from about 20% and/or 30% and/or 40% and/or 45% and/or 50% to about 75% and/or 80% and/or 85% and/or 90% by weight based on the total weight of the filament-forming composition. The non-volatile components may be composed of filament-forming materials, such as backbone polymers, active agents and combinations thereof. Volatile components of the filament-forming composition will comprise the remaining percentage and range from 10% to 80% by weight based on the total weight of the filament-forming composition.

For successful fiber spinning of complex mixtures, such as molten fatty alcohols or aqueous surfactant solutions it is generally necessary to add a polymeric ingredient called a structurant. The structurant's purpose is to increase the shear and extensional viscosity of the fluid to enable fiber formation. The structurant is generally a high molecular weight species, usually in the 100,000-6,000,000 g/mol range. However, a balance is often struck between concentration and molecular weight, such that when a lower molecular weight species is used, it requires a higher level to function properly. Likewise, when a higher molecular species is used, lower levels can be used to enable fiber spinning. An important aspect of the structurant is its solubility in the spinning fluid to enable viscosity build for fiber formation. The structurants polyvinylpyrrolidone and polyethylene oxide have been found to be two such polymers that meet the criteria of solubility in the spinning fluid and and capable of being produced at high molecular weights.

### b. Spinning Operation (20)

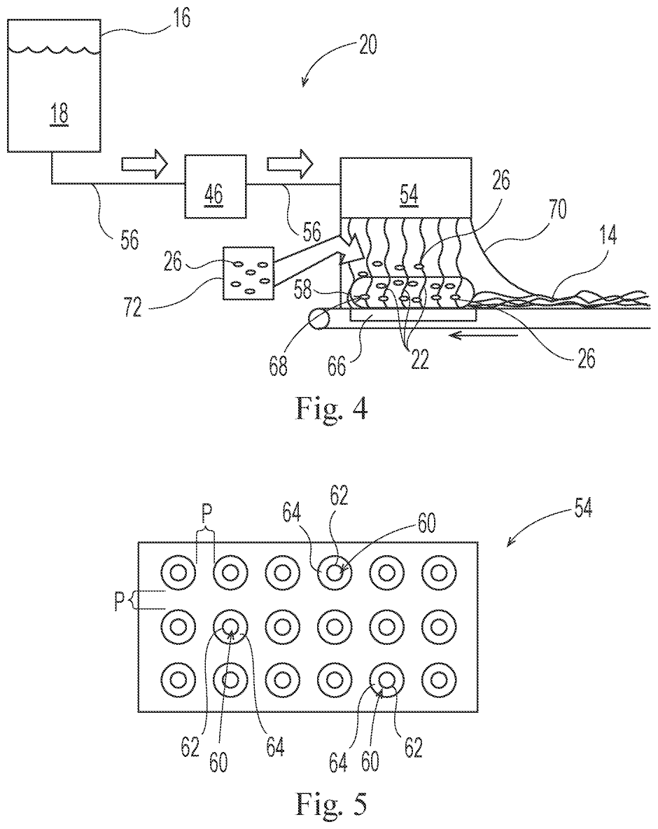

The filaments 22 of the present invention comprising one or more filament-forming materials 18 and optionally, one or more active agents 48, present within the filament 22 may be made as shown in FIGS. 4 and 5. As shown in FIGS. 4 and 5, a spinning operation 20 for making a filaments 22 from a filament-forming composition 18, in a continuous process, according to the present invention comprises the steps of:

a. providing a filament-forming composition 18 delivered to the spinning operation 20 from a filament-forming composition making operation 16, wherein the filament-forming composition 18 comprises one or more filament-forming materials 38 and optionally, one or more active agents 48 and/or one or more polar solvents (such as water), and optionally one or more deterrent agents; and

b. spinning the filament-forming composition 18, such as via a spinning die 54, for example a multi-row capillary spinning die, such as a Biax-fiberfilm multi-row capillary die, into one or more filaments 22 comprising the one or more filament-forming materials 38 and optionally, the one or more active agents 48 and the one or more deterrent agents.

The filament-forming composition 18 may be processed (spun) from the spinning die 54 at a temperature of from about 20° C. to about 100° C. and/or from about 30° C. to about 90° C. and/or from about 35° C. to about 70° C. and/or from about 40° C. to about 60° C. when making filaments 22 from the filament-forming composition 18.

The filament-forming composition 18 may be transported via suitable piping 56, with or without a pump 46, from the filament-forming composition making operation 16 to the spinning die 54. A pump 46, such as a Zenith®, H-9000, having a capacity of 30 and/or 45 cubic centimeters per revolution (cc/rev), manufactured by Colfax Corporation, Zenith Pumps Division, of Monroe, N.C., USA may be used to facilitate transport of the filament-forming composition 18 to a spinning die 54. The flow of the filament-forming composition 18 from the filament-forming composition making operation 16 to the spinning die 54 may be controlled by adjusting the number of revolutions per minute (rpm) of the pump 46.

The filaments 22 spun from the spinning die 54 may be collected, for example continuously onto a collection device 58, such as a belt and/or fabric, for example a patterned belt, and/or a rotary drum, that is continuously operating to move the collected filaments 22, which form a fibrous composition 14, such as a plurality of inter-entangled filaments, on the collection device 58 further down the process to other operations in the making of the article of manufacture 12 of the present invention.

The total level of the one or more filament-forming materials present in the fibrous element 10, when active agents are present therein, may be less than 80% and/or less than 70% and/or less than 65% and/or 50% or less by weight on a dry fibrous element basis and/or dry soluble fibrous composition basis and the total level of the one or more active agents, when present in the fibrous element may be greater than 20% and/or greater than 35% and/or 50% or greater 65% or greater and/or 80% or greater by weight on a dry fibrous element basis and/or dry soluble fibrous composition basis.

As shown in FIGS. 4 and 5, the spinning die 54 may comprise a plurality of filament-forming holes that include a melt capillary 34 encircled by a concentric attenuation fluid hole 36 through which a fluid, such as air, passes to facilitate attenuation of the filament-forming composition 22 into a fibrous element 10 as it exits the filament-forming hole 32.

In one example, the spinning die 54 shown in FIG. 5 has two or more rows of circular extrusion nozzles (filament-forming holes 60) spaced from one another at a pitch P of about 1.524 millimeters (about 0.060 inches). The nozzles have individual inner diameters of about 0.305 millimeters (about 0.012 inches) and individual outside diameters of about 0.813 millimeters (about 0.032 inches). Each individual nozzle comprises a melt capillary 62 encircled by an annular and divergently flared orifice (concentric attenuation fluid hole 64) to supply attenuation air to each individual melt capillary 62. The filament-forming composition 18 extruded through the extrusion nozzles (filament-forming holes 60) is surrounded and attenuated by generally cylindrical, humidified air streams supplied through the orifices to produce filaments 22.

Attenuation air can be provided by heating compressed air from a source by an electrical-resistance heater, for example, a heater manufactured by Chromalox, Division of Emerson Electric, of Pittsburgh, Pa., USA.

The embryonic filaments 22 are dried by a drying air stream having a temperature from about 149° C. (about 300° F.) to about 315° C. (about 600° F.) by an electrical resistance heater and/or a gas burner (direct or indirect) (not shown) supplied through drying nozzles and discharged at an angle of about 90° relative to the general orientation of the embryonic filaments 22 being spun. The dried filaments 22 may be collected on a collection device 58, such as a belt or fabric, in one example a belt or fabric capable of imparting a pattern, for example a non-random repeating pattern to a fibrous composition 14, such as a soluble fibrous composition, formed as a result of collecting the filaments 22 on the belt or fabric. The addition of a vacuum source 66 directly under a formation zone 68, the area on the collection device 58 where the filaments 22 contact the collection device 58, may be used to aid collection of the filaments 22 on the collection device 58. The spinning and collection of the filaments 22 produce a fibrous composition 14, for example a soluble fibrous composition, comprising inter-entangled filaments.

In one example, a spinning enclosure 70, which is a housing that at least partially encloses, in one example fully encloses to the extent that the collection device 58 and fibrous composition 14 carried on the collection device 58 are able to move freely under the spinning enclosure 70, the filaments 22 being spun from the spinning die 54 to the collection device 58. The spinning enclosure 70 at least partially controls the environment that the filaments 22 are exposed to down the spinline from the spinning die 54 to the collection device 58.

In one example, during the spinning step, any volatile solvent, such as water, present in the filament-forming composition 18 is removed, such as by drying, as the filament 22 is formed. In one example, greater than 30% and/or greater than 40% and/or greater than 50% of the weight of the filament-forming composition's 18 volatile solvent, such as water, is removed during the spinning step, such as by drying the filament 22 being produced.

In one example, the filaments 22 are spun from one spinning die 54, for example a multi-row capillary die.

In one example, two or more different filaments 22 are spun from at least one spinning die 54 (the same spinning die 54).

In one example, the filaments 22 are spun from two or more spinning dies 54.

In one example, the process of the present invention may comprise two or more spinning operations 20. In one example, a first spinning operation 20 comprises spinning filaments 22 from a filament-forming composition 18 comprising one or more filament-forming materials 38 with or without active agents 48 and without the inclusion of solid additives, for example particles 26 via a commingling operation 24, to produce a fibrous composition 14 on a collection device 58, which may be the same collection device 58 upon which the filaments 22 from a second spinning operation 20 are collected. A second spinning operation 20 downstream of the first spinning operation 20 comprises spinning filaments 22 from a filament-forming composition 18 comprising one or more filament-forming materials 38 with or without active agents 48 and with the inclusion of solid additives, for example particles 26 via a commingling operation 24, onto the fibrous composition 14 formed by the first spinning operation 20.

The filament-forming composition 18 may comprise any suitable total level of filament-forming materials 38 and any suitable level of active agents 48 so long as the filament 22 produced from the filament-forming composition 18 comprises a total level of filament-forming materials 38 in the filament 22 of from about 5% to 100% or less by weight on a dry filament basis and/or dry soluble fibrous composition basis and a total level of active agents 48 in the filament 22 of from 0% to about 95% by weight on a dry filament basis and/or dry soluble fibrous composition basis.

### c. Commingling Operation (24)

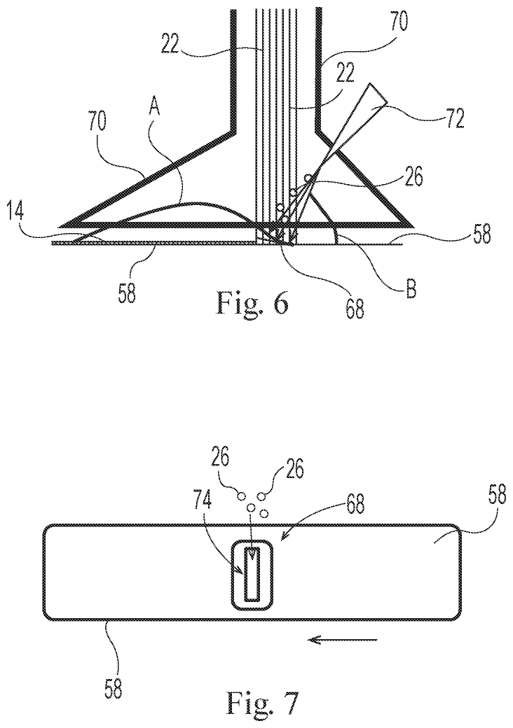

In one example, as shown in FIG. 6, particles 26 may be added to the filaments 22 being spun from the spinning die 54 within the spinning enclosure 70. The addition of particles 26 may be accomplished during the formation of the filaments 22 and/or after collection of the filaments 22 on the collection device 58. The particles 26 may be added into the fibrous composition 14 and/or filaments 22 from a particle source 72. The particles 26 may be added such that the particles 26 are collected on a surface of the collection device 58 inside the spinning enclosure 70. The collection device 58 may be operable within the formation zone 68, which may be inside the spinning enclosure 70. The spinning enclosure 70 may be positioned above the collection device 58 and encompass the formation zone 68 on the collection device 58.

The addition of particles 26 may result in said particles 26 being entrapped and/or entrained within the filaments 22 and/or fibrous composition 14 collected on the collection device 58.

A particle source 72, for example a feeder, suitable to supply a flow of particles is placed directly above the drying region for the fibrous elements as shown in FIG. 11. In this case for example a vibratory feeder made by Retsch® of Haan, Germany, is used. In order to aid in a consistent distribution of particles in the cross direction the particles are fed onto a tray (not shown) that started off the width of the particle source 72 and ended at the same width as the spinning die 54 face to ensure particles 26 are delivered into all areas of filament 22 formation. The tray is completely enclosed with the exception of the exit to minimize disruption of the particle feed.

While filaments 22 are being formed, the particle source 72 is turned on and particles 26 are introduced into the filament 22 stream. The particles 26 are commingled with the filaments 22 within the spinning enclosure 70. The commingled filaments 22 and particles 26 are collected on the collection device 58 as a composite structure (filaments 22 and particles 26 commingled together). The composite structure is referred to as a fibrous composition 14.

The particles 26 may be introduced into the spinning enclosure 70 between the spinning die 54 and the collection device 58, as shown in FIGS. 4 and 6, at any angle so long as at least a portion of the particles 26 contact the filaments 22 at the formation zone 68. As shown in FIG. 6, if the introduction of the particles 26 into the filament 22 stream is not tailored to result in the particles 26 contacting the filaments 22 in the formation zone 68 then the particles may end up downstream (points in the process that are closer to the finished article of manufacture and/or packaged article of manufacture relative to the referenced point, for example if the reference point is the spinning operation or collecting operation, then downstream means converting operation and/or packaging operation) of the formation zone 68 as shown by the particle trajectory line A in FIG. 6 and/or upstream (points in the process that are farther away from the finished article of manufacture 12 and/or packaged article of manufacture 32 relative to the referenced point, for example if the reference point is the spinning operation or collecting operation, then upstream means, for example the filament-forming composition making operation 16) of the formation zone 68 as shown by the particle trajectory line B in FIG. 6.

In one example, the solid additives, for example particles 26, contact the filaments 22 on the upstream side of the spinning enclosure 70.

In another example, the solid additives, for example particles 26, contact the filaments 22 on the downstream side of the spinning enclosure 70.

In another example, the solid additives, for example particles 26, contact the filaments 22 on both the upstream and downstream sides of the spinning enclosure 70.

FIG. 7 illustrates schematically another example of the commingling operation 24 wherein the particles 26 land on the collection device 58 in a particle landing zone 74 and contact the filaments 22 within the formation zone 68.

The solid additives, for example particles 26, may contact the filaments 22 at a contact angle (the contact angle is relative to the filament stream direction emanating and exiting from the spinning die 54) of greater than or equal to about 0° but less than or equal to about 90° and/or greater than or equal to about 10° but less than or equal to about 90° and/or greater than or equal to about 20° but less than or equal to about 90° and/or greater than or equal to about 30° but less than or equal to about 90° and/or at least about 40° but less than about 90° and/or at a contact angle of at least about 45° but less than about 90°.

The solid additives, for example particles 26, may be dispersed throughout the fibrous composition 14 at an overall MD basis weight variation % RSD of less than 40.0% and/or less than 30.0% and/or less than 25.0% and/or less than 20.0% and/or less than 15.0% and/or less than 10.0% and/or less than 5.0% and/or about 0% as measured according to the CD and MD Basis Weight Variation Test Method described herein.

The solid additives, for example particles 26, may be dispersed throughout the fibrous composition 14 at an overall CD basis weight variation % RSD of less than 40.0% and/or less than 30.0% and/or less than 25.0% and/or less than 20.0% and/or less than 15.0% and/or less than 10.0% and/or less than 5.0% and/or about 0% as measured according to the CD and MD Basis Weight Variation Test Method described herein.

The solid additives, for example particles 26, may contact the filaments at a velocity of greater than 1 m/s and/or at least 2 m/s and/or at least 2.5 m/s and/or less than 10 m/s and/or less than 8 m/s and/or 6 m/s or less and/or from about 1 m/s to about 20 m/s.

The solid additives, for example particles 26, may be commingled with the filaments 22 such that a solid additive inclusion efficiency (for example particle inclusion efficiency) of greater than 40% and/or at least 42% and/or at least 45% and/or at least 50% and/or at least 54% and/or at least 65% and/or at least 75% and/or at least 85% and/or at least 90% and/or at least 95% and/or at least 98% as measured according to the Inclusion Efficiency Test Method described herein.

In one example, the step of commingling comprises introducing the solid additives, for example particles 26, into the plurality of filaments 22, for example soluble filaments, between at least one of the spinning dies 54 and the collection device 58. In one example, the solid additives, for example particles 26, are introduced more proximal to the at least one spinning die 54 than to the collection device 58.

In one example, the commingling operation (step) comprises introducing the solid additives, for example particles 26, into the filaments 22, for example soluble filaments, spun from two different spinning dies 54.

The solid additives, for example particles 26, may comprise one or more types or different types of particles 26. In one example, the solid additives, for example particles 26, comprise a mixture of particles 26 of differing compositions. In another example, the solid additives, for example particles 26, comprise a blend of particle of differing composition. In another example, the solid additives, for example particles 26, comprise water-soluble particles and/or water-insoluble particles, which may comprise water-swellable particles. Further, in one example, the particles 26 may be in the form of an agglomerate, for example an agglomerate comprising a water-soluble material and a water-insoluble material.

In one example, the solid additives, for example particles 26, may exhibit a D50 particle size of from about 100 μm to about 5000 μm and/or from about 100 μm to about 2000 μm and/or from about 250 μm to about 1200 μm and/or from about 250 μm to about 850 μm as measured according to the Particle Size Distribution Test Method described herein.

In one example, the solid additives, for example particles 26, may exhibit a D10 of 250 μm as measured according to the Particle Size Distribution Test Method described herein.

In another example, the solid additives, for example particles 26, may exhibit a D90 of 1200 μm and/or 850 μm as measured according to the Particle Size Distribution Test Method described herein.

In one example, the solid additives, for example particles 26, may exhibit a D10 of greater than 44 μm and/or greater than 90 μm and/or greater than 150 μm and/or greater than 212 μm and/or greater than 300 μm as measured according to the Particle Size Distribution Test Method described herein.

In one example, the solid additives, for example particles 26, may exhibit a D90 of less than 1400 μm and/or less than 1180 μm and/or less than 850 μm and/or less than 600 μm and/or less than 425 μm as measured according to the Particle Size Distribution Test Method described herein.

In one example, the solid additives, for example particles 26, may exhibit any combination of the above-identified D10, D50, and/or D90 so long as D50, when present, is greater than D10, when present, and D90, when present, is greater than D10 and D50, when present.

In one example, the solid additives, for example particles 26, may exhibit any combination of the above-identified D10 and D90 so long as D90 is greater than D10.

In one example, the solid additives, for example particles 26, may exhibit a D10 of greater than 212 μm and a D90 of less than 1180 μm as measured according to the Particle Size Distribution Test Method described herein.

In one example, the solid additives, for example particles 26, may exhibit a D10 of greater than 90 μm and a D90 of less than 425 μm as measured according to the Particle Size Distribution Test Method described herein.

In one example, the spinning operation 20 may comprise two or more spinning dies 54 arranged adjacent to each other in the machine direction and/or in the cross-machine direction. In one example, when the spinning operation 20 comprises two or more dies arranged adjacent to each other in the machine direction, a commingling operation 24 may be positioned between two adjacent (in the machine direction) spinning dies 54.

The particles 26 used in the present invention for commingling with the filaments 22 may be active agent-containing particles.

### d. Collecting Operation (28)

As shown in FIGS. 1, 4, and 6, the filaments 22 from the spinning operation 20, and optionally the solid additives, for example particles 26, from the commingling operation 24, are collected on a collection device 58 during the collecting operation 28 to form a fibrous composition 14, which may be a composite structure (commingled filaments 22 and particles 26).

In one example, the collection device 58 may be a belt, such as a patterned belt that imparts a texture, such as a three-dimensional texture to at least one surface of the fibrous composition 14 and/or a rotary drum. The collection device 58 may impart a pattern, for example a non-random, repeating pattern which may be continuous, discontinuous, and/or semi-continuous in nature. The collection device 58 may create different regions within the fibrous composition 14, for example different average densities.

### e. Depression Forming Operation (29)

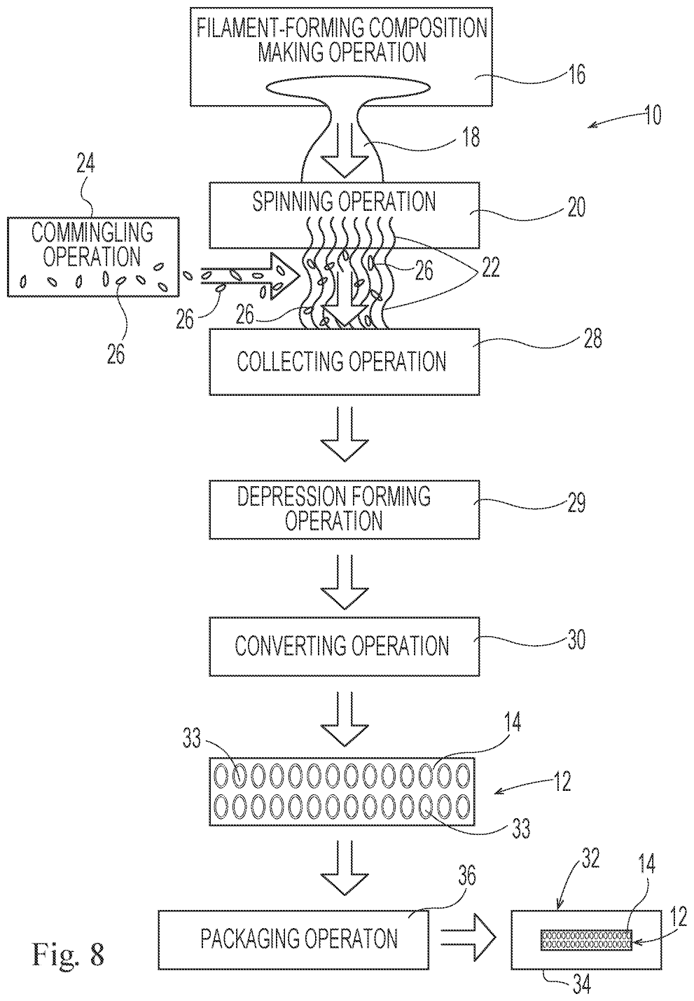

As shown in FIG. 8, pressure can be applied to the fibrous composition 14, which may be a composition structure (commingled filaments 22 and particles 26), to create a depression 33 suitable for the addition of a nonfibrous composition. The depression 33 in the fibrous composition 14 is larger in volume than the three-dimensional texture imparted by the collection 58.

The depression forming operation 29 can be a step in the continuous process, as described herein and shown in FIG. 8, or a discrete step, separate from the continuous process.

The depression forming operation 29 can be performed using any suitable means, such as for example, manual depression by hand or other suitable tool, mechanical embossing, a vacuum pull of the fibrous composition into a die plate, a vacuum pull of a separate sheet of material in contact with the fibrous composition, collecting the spun fiber on a embossed/debossed belt or surface, a positive air pressure applied to the fibrous composition into a die plate, a positive air pressure applied to a separate sheet of material in contact with the fibrous composition, and/or combinations thereof.

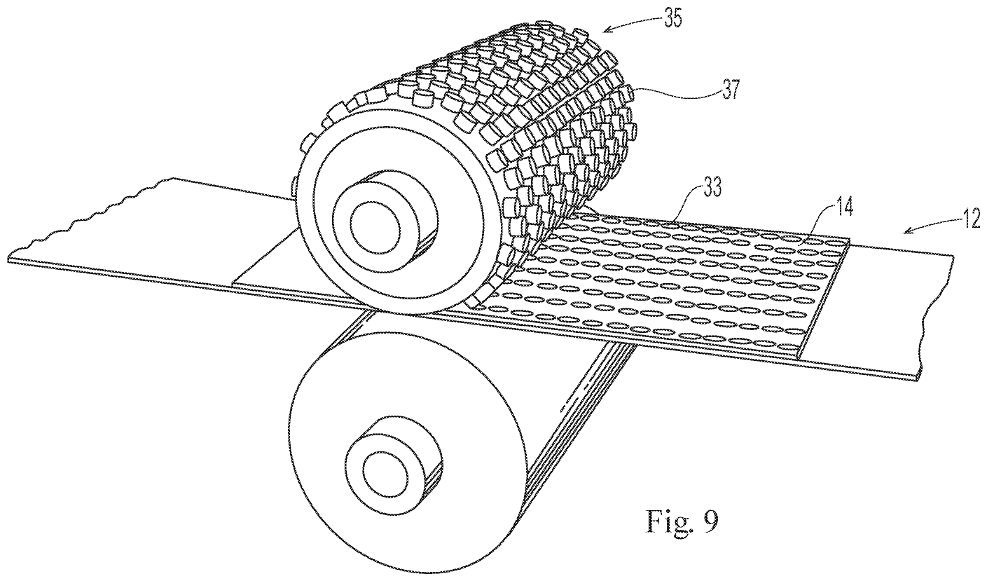

Mechanical embossing can be accomplished by applying an embossed die plate directly to the fibrous composition 14, by applying a debossed die directly to the fibrous composition 14, and/or combinations thereof. The die plate can be applied as a perpendicular or rotary press to the fibrous composition 14, such as the rotary press 35 in FIG. 9 showing individual raised elements 37 to create the depression 33 in the fibrous composition 14.

The moisture of the fibrous composition 14 can be modified to increase the flexibility of the fibrous composition 14 to survive the depression forming operation 29. For example, if the fibrous composition 14 does not have enough moisture, the fibrous composition 14 may fracture during the depression forming operation 29. The fibrous composition 14 can be stored at a humidity at from about 20% to about 60%, from about 25% to about 55%, or from about 30% to about 50% to raise the moisture content of the fibrous composition to allow for the depression forming composition 29 to be successful without leading to cracking or fracturing.

Heat can be applied to the fibrous composition 14 during and/or before the depression forming operation 29 in order to improve the depression forming operation 29. The web tension of the fibrous composition 14 can be increased or decreased to improve the depression forming operation 29.

A perforation or fiber slit can be added to the fibrous composition 14 near the location of the depression 33 to allow the fibrous composition 14 to be more easily deformed into the emboss cavity.

### f. Converting Operation (30)

As shown in FIGS. 1 and 8, the fibrous composition 14, which may be a composition structure (commingled filaments 22 and particles 26), can be converted into an article of manufacture. Thus, the converting operation 30, as described herein, can include one or more steps for converting the fibrous composition 14 into an article of manufacture. For example, the converting operation can include slitting, stacking, calendaring, treating with a nonfibrous composition, die-cutting, printing, and/or combinations thereof.

The nonfibrous composition can be a liquid, paste, or solid composition comprising an abrasive, whitening agents, flavoring agents, effervescent agents, and the like. The nonfibrous composition can include components of the article of manufacture that can disrupt the continuous process, or ones that would be degraded or impacted due to the continuous process.

For example, a solid particle of a suitable particle size, as described herein, can clog the die capillaries. These solid particles, such as an abrasive, can be added during a comingling operation 24 or the converting operation. Additionally, many oral care active agents can be degraded at the temperatures needed to spin filaments in the filament-forming composition making operation 16. These oral care active agents, such as whitening agents, flavoring agents, effervescent agents, and/or any other oral care active agent which is impacted during the spinning operation can be added during the commingling operation 24 or the converting operation 30, as a nonfibrous composition.

### Test Methods

Unless otherwise specified, all tests described herein including those described under the Definitions section and the following test methods are conducted on samples that have been conditioned in a conditioned room at a temperature of 23° C.±1.0° C. and a relative humidity of 50%±2% for a minimum of 2 hours prior to the test. The samples tested are “usable units.” “Usable units” as used herein means sheets, flats from roll stock, pre-converted flats, and/or single or multi-ply products. All tests are conducted under the same environmental conditions and in such conditioned room. Do not test samples that have defects such as wrinkles, tears, holes, and like. Samples conditioned as described herein are considered dry samples (such as “dry filaments”) for testing purposes. All instruments are calibrated according to manufacturer's specifications.

### Basis Weight Test Method

Basis weight is defined as the weight in g/m² of a sample being tested. It is determined by accurately weighing a known area of a conditioned sample using an appropriate balance, recording the weight and area of sample tested, applying the appropriate conversion factors, and finally calculating the basis weight in g/m² of the sample.

Basis weight is measured by cutting a sample from a single web, a stack of webs, or other appropriate plied up, or consumer salable unit and weighing the sample using a top loading analytical balance with a resolution of ±0.001 g. The sample must be equilibrated at a temperature of 73°±2° F. (23°±1° C.) and a relative humidity of 50% (±2%) for a minimum of two hours prior to cutting samples. During weighing, the balance is protected from air drafts and other disturbances using a draft shield. A precision cutting die, measuring 1.625×1.625 in (41.275×41.275 mm) is used to prepare all samples. Select usable sample areas which are clean, free of holes, tears, wrinkles and other defects.

For each sample use the die cutter described above to cut a sample, weigh the mass of the sample, and record the mass result to the nearest 0.001 g.

The Basis Weight is calculated in g/m2 as follows:

Basis Weight=(Mass of sample)/(Area of sample).

Or specifically,

Basis Weight (g/m2)=(Mass of sample (g))/(0.001704 m2).

Report result to the nearest 0.1 g/m2. Sample dimensions can be changed or varied using a similar precision cutter as mentioned above. If the sample dimension is decreased, then several samples should be measured and the mean value reported as its basis weight.

Particle Size Distribution Test Method: The particle size distribution test is conducted to determine characteristic sizes of solid additives, for example particles. It is conducted using ASTM D 502-89, “Standard Test Method for Particle Size of Soaps and Other Detergents”, approved May 26, 1989, with a further specification for sieve sizes and sieve time used in the analysis. Following section 7, “Procedure using machine-sieving method,” a nest of clean dry sieves containing U.S. Standard (ASTM E 11) sieves #4 (4.75 mm), #6 (3.35 mm), #8 (2.36 mm), #12 (1.7 mm), #16 (1.18 mm), #20 (850 micrometer), #30 (600 micrometer), #40 (425 micrometer), #50 (300 micrometer), #70 (212 micrometer), #100 (150 micrometer), #170 (90 micrometer), #325 (44 micrometer) and pan is required to cover the range of particle sizes referenced herein. The prescribed Machine-Sieving Method is used with the above sieve nest. A suitable sieve-shaking machine can be obtained from W.S. Tyler Company, Ohio, U.S.A. The sieve-shaking test sample is approximately 100 grams and is shaken for 5 minutes.

The data are plotted on a semi-log plot with the micrometer size opening of each sieve plotted on the logarithmic abscissa and the cumulative mass percent finer (CMPF) is plotted on the linear ordinate. An example of the above data representation is given in ISO 9276-1:1998, “Representation of results of particle size analysis—Part 1: Graphical Representation”, Figure A.4. A characteristic particle size (Dx, x=10, 50, 90), for the purpose of this invention, is defined as the abscissa value at the point where the cumulative mass percent is equal to x percent, and is calculated by a straight line interpolation between the data points directly above (a) and below (b) the x value using the following equation:

Dx=10\hat{ }[Log(Da)−(Log(Da)−Log(Db))*(Qa−x%)/(Qa−Qb)]

where Log is the base 10 logarithm, Qa and Qb are the cumulative mass percentile values of the measured data immediately above and below the x^{th} percentile, respectively; and Da and Db are the micrometer sieve size values corresponding to these data. Example data and calculations:

| sieve size | weight on | cumulative mass % |
|---|---|---|
| (micrometer) | sieve (g) | finer (CMPF) |
| 1700 | 0 | 100% |
| 1180 | 0.68 | 99.3% |
| 850 | 10.40 | 89.0% |
| 600 | 28.73 | 60.3% |
| 425 | 27.97 | 32.4% |
| 300 | 17.20 | 15.2% |
| 212 | 8.42 | 6.8% |
| 150 | 4.00 | 2.8% |
| Pan | 2.84 | 0.0% |

For D10 (x=10), the micrometer screen size where CMPF is immediately above 10% (Da) is 300 micrometer, the screen below (Db) is 212 micrometer. The cumulative mass immediately above 10% (Qa) is 15.2%, below (Qb) is 6.8%. D10=10\hat{ }[Log(300)−(Log(300)−Log(212))*(15.2%−10%)/(15.2%−6.8%)]=242 micrometer.

For D90 (x=90), the micrometer screen size where CMPF is immediately above 90% (Da) is 1180 micrometer, the screen below (Db) is 850 micrometer. The cumulative mass immediately above 90% (Qa) is 99.3%, below (Qb) is 89.0%. D90 =10\hat{ }[Log(1180)−(Log(1180)−Log(850))*(99.3%−90%)/(99.3%−89.0%]=878 micrometer. For D50 (x=50), the micrometer screen size where CMPF is immediately above 50% (Da) is 600 micrometer, the screen below (Db) is 425 micrometer. The cumulative mass immediately above 50% (Qa) is 60.3%, below (Qb) is 32.4%. D50 =10\hat{ }[Log(600)−(Log(600)−Log(425))*(60.3%−50%)/(60.3%−32.4%)]=528 micrometer.

### CD and MD Basis Weight Variation Test Method

The cross direction (CD) basis weight variation is measured with this method by sampling the web in the cross direction at a given fixed machine direction (MD) position, measuring the basis weight for samples taken at this MD position, and then calculating the % Relative Standard Deviation (RSD) for the sample set. This analysis is performed for as many samples as needed to sample the entire cross direction of a given web. As one sampling example, if the web is about 53 cm wide and the sample die cutter for basis eight is 4.1275 cm wide as it is in the Basis Weight Method described herein, then about 12 samples may be taken across the web. Samples at the web edges may not completely fill the sampling die when cutting across a full MD position, for example, the die cutter extends past the edge of the web, should be discarded. Sampling at a given MD position may vary slightly, as long as the entire CD width is reasonably sampled at the respective MD position. The sampling is completed for a total of 10 fixed MD positions spaced about 1 m apart. The CD basis weight variation is recorded for each MD position and the values at each position are used to get a CD basis weight variation % RSD per MD sampled position. The average for the 10 rows or MD positions sampled is reported as the overall CD basis weight variation % RSD.

The machine direction (MD) basis weight variation is measured with this method by sampling the web in the Machine Direction at a given fixed cross direction (CD) position, measuring the basis weight for samples taken at the given CD position, replicating the measurement at other CD positions, and then calculating the overall MD basis weight variation % RSD for the entire sample set.

The overall CD basis weight variation % RSD and overall MD basis weight variation % RSD can be averaged to get an overall web basis weight variation % RSD.

### Procedure for Measuring Cross Direction Variability at Fixed Machine Direction Position

Choose a Machine Direction position of the web from which to sample.

Follow the Basis Weight Test Method described herein to measure the basis weigh of all samples.

Cut as many samples as necessary to sample the entire web width at a given MD position.

As one example, if a web is about 53 cm wide and the sample die cutter is 4.1275 cm wide, then about 12 samples may be taken across the web. Sampling at a given MD position may vary slightly, as long as the entire CD width is reasonably sampled at the respective MD position.

Discard samples at the edges of the full web that do not completely fill the sampling die when cutting.

Calculate the basis weight for each sample taken along the given MD position.

Calculate the mean sample basis weight for this fixed MD position.

Calculate the standard deviation for the samples at the fixed MD position.

Calculate the % RSD (Relative Standard Deviation) for the samples at this MD position by dividing the standard deviation by the mean sample basis weight and multiply by 100 to yield a % value.

Repeat the above for a total of 10 rows or 10 MD positions, sampling at about 1 meter intervals of the web from the process.

Average the % RSD for all ten rows and report it as the overall CD basis weight variation % RSD. This value is reported to the nearest 0.1%.

### Procedure For Measuring Machine Direction Variability at Fixed Cross Direction Position

Sample from the web at its cross direction centerline position.

Follow the Basis Weight Test Method described herein to measure the basis weight of all samples.

Cut ten samples along the web's cross direction centerline position at about 1 m intervals in the MD direction of the web from the process.

Calculate the basis weight for each sample.

Calculate the mean sample basis weight for CD centerline position.

Calculate the standard deviation for the same sample set.

Calculate the MD basis weight variation % RSD at the CD centerline position by dividing the standard deviation by the mean sample basis weight and multiplying by 100 to get a % value. Report this value to the nearest 0.1%.

Repeat the above cross direction centerline position measurement by doing the same sampling and measurement along a mid-line on the left half of the CD centerline and then along a mid-line on the right half of the CD centerline.

From the above analysis, there will be three values generated:

% RSD for the CD centerline position

% RSD for the mid-line on the left half of the CD centerline

% RSD for the mid-line on the right half of the CD centerline

Average the % RSD for these three CD positions and report it as the overall MD basis weight variation % RSD. This value is reported to the nearest 0.1%.

### Inclusion Efficiency Test Method

Inclusion efficiency is a measure of the percentage of solid additives, for example particles, captured and retained in the fibrous composition during the commingling (coforming) operation to the number of solid additives, for example particles, introduced (fed) into the commingling (coforming) operation. A higher percentage of inclusion efficiency indicates better solid additive, for example particle, entrainment is being realized by the commingling (coforming) operation and/or coforming apparatus and/or process conditions operable during the commingling (coforming) operation.

In general, the inclusion efficiency is:

$$Inclusion Efficiency = \frac{\begin{matrix}Ratio of mass of particles to mass of \\ filaments in fibrous composition X\end{matrix}}{100}$$

Ratio of mass of particles to mass of dry filament feed rate which is better calculated as follows:

$$Inclusion Efficiency = 100 X \frac{\frac{\begin{matrix}( ( Composite fibrous composition \\ basis weight ( g / {m}^{2} ) - Filament \\ fibrous structure ( void of solid \\ additives ) basis weight ( g / {m}^{2} ) )\end{matrix}}{\begin{matrix}( Filament fibrous composition \\ ( void of solid additives ) basis \\ weight ( g / {m}^{2} ) ) )\end{matrix}}}{\begin{matrix}( Total Particle feed rate ( g / min ) ) / \\ ( Total Filament - forming \\ composition feed rate ( g / min ) X \\ Filament - forming composition \\ Solids concentration )\end{matrix}}$$

### Procedure

Run the commingling (coforming) operation at steady state conditions to make a base fibrous composition (filament only) with no particles.

Measure the basis weight of cut sample of base fibrous composition as defined in the Basis Weight method defined herein.

Sample from the center of the base fibrous composition cross direction or at the base fibrous composition's CD centerline.

Record this as the base fibrous composition basis weight (g/m²).

Make composite fibrous composition (filaments+particles) at a desired dry mass feed rate.

Measure the basis weight of cut sample of the composite fibrous composition as defined in the Basis Weight method defined herein.

Sample from the center of the composite fibrous composition cross direction or at the composite fibrous composition's CD centerline.

Record this as the composite fibrous composition basis weight (g/m²).

Total Particle feed rate and Total Filament-forming Composition feed rate are process parameters. Total Particle feed rate is measured by collecting the entire particle feed stream over a one minute interval and reported in g/min to the nearest 1 g/min Total Filament-forming Composition feed rate is measured using inline process flow meters and is reported in g/min to the nearest 1 g/min Filament-forming Composition Solids concentration is the ratio of the mass of filament-forming composition material left after drying to the mass of starting filament-forming composition. This can be measured using a Mettler Toledo HC103 or equivalent Moisture Analyzer. Filament-forming Composition Solids concentration is reported as a fractional value to the nearest 0.01 units or as a percentage to the nearest 1%.

For clarity, an example of an Inclusion Efficiency calculation is shown below.

A base fibrous composition (filament only—no particles) is made from a filament-forming composition at 55% (0.55) solids concentration. The filament-forming composition feed rate to the die is 1600 g/min. A sample cut the base fibrous composition's centerline exhibits a basis weight of 264 g/m². A composition fibrous composition (filaments+solid additives, for example particles) is then made as described above with respect to the base fibrous composition, but the solid additives, for example particles, are added to the filaments at a Total Particle feed rate of 2350 g/min A sample cut from the composite fibrous composition exhibits a basis weight 870 g/m². With these values, the example Inclusion Efficiency calculation is as follows:

$$Inclusion Efficiency = \frac{( 870 g / {m}^{2} - 264 g / {m}^{2} ) / ( 264 g / {m}^{2} )}{( 2350 g / min ) / ( 1600 g / min * 0.55 )} \times 100 = 86 \%$$

Inclusion Efficiency is reported to the nearest 1%.

### Water Content Test Method

The water (moisture) content present in a fibrous element and/or particle and/or fibrous composition is measured using the following Water Content Test Method. A fibrous element and/or particle and/or fibrous composition or portion thereof (“sample”) in the form of a pre-cut sheet is placed in a conditioned room at a temperature of 23° C.±1.0° C. and a relative humidity of 50%±2% for at least 24 hours prior to testing. Each fibrous composition sample has an area of at least 4 square inches, but small enough in size to fit appropriately on the balance weighing plate. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the samples are placed into the forced air oven on top of foil for 24 hours at 70° C.±2° C. at a relative humidity of 4% 35 2% for drying. After the 24 hours of drying, the sample is removed and weighed within 15 seconds. This weight is designated as the “dry weight” of the sample.

The water (moisture) content of the sample is calculated as follows:

$$\% Water in sample = 100 \% \times \frac{( Equilibrium weight of sample - Dry weight of sample )}{Dry weight of sample}$$

The % Water (moisture) in sample for 3 replicates is averaged to give the reported % Water (moisture) in sample. Report results to the nearest 0.1%.

### Diameter Test Method

The diameter of a discrete fibrous element or a fibrous element within a fibrous composition is determined by using a Scanning Electron Microscope (SEM) or an Optical Microscope and an image analysis software. A magnification of 200 to 10,000 times is chosen such that the fibrous elements are suitably enlarged for measurement. When using the SEM, the samples are sputtered with gold or a palladium compound to avoid electric charging and vibrations of the fibrous element in the electron beam. A manual procedure for determining the fibrous element diameters is used from the image (on monitor screen) taken with the SEM or the optical microscope. Using a mouse and a cursor tool, the edge of a randomly selected fibrous element is sought and then measured across its width (i.e., perpendicular to fibrous element direction at that point) to the other edge of the fibrous element. A scaled and calibrated image analysis tool provides the scaling to get actual reading in um. For fibrous elements within a fibrous composition, several fibrous element are randomly selected across the sample of the fibrous composition using the SEM or the optical microscope. At least two portions of the fibrous composition are cut and tested in this manner Altogether at least 100 such measurements are made and then all data are recorded for statistical analysis. The recorded data are used to calculate average (mean) of the fibrous element diameters, standard deviation of the fibrous element diameters, and median of the fibrous element diameters.

Another useful statistic is the calculation of the amount of the population of fibrous elements that is below a certain upper limit. To determine this statistic, the software is programmed to count how many results of the fibrous element diameters are below an upper limit and that count (divided by total number of data and multiplied by 100%) is reported in percent as percent below the upper limit, such as percent below 1 micrometer diameter or %-submicron, for example. We denote the measured diameter (in μm) of an individual circular fibrous element as di.

In the case that the fibrous elements have non-circular cross-sections, the measurement of the fibrous element diameter is determined as and set equal to the hydraulic diameter which is four times the cross-sectional area of the fibrous element divided by the perimeter of the cross-section of the fibrous element (outer perimeter in case of hollow fibrous elements). The number-average diameter, alternatively average diameter is calculated as:

$${d}_{num} = \frac{{\sum }_{i = 1}^{n} {d}_{i}}{n}$$

### Weight Average Molecular Weight

The weight average molecular weight (Mw) of a material, such as a polymer, is determined by Gel Permeation Chromatography (GPC) using a mixed bed column. A high performance liquid chromatograph (HPLC) having the following components: Millenium®, Model 600E pump, system controller and controller software Version 3.2, Model 717 Plus autosampler and CHM-009246 column heater, all manufactured by Waters Corporation of Milford, Mass., USA, is utilized. The column is a PL gel 20 pm Mixed A column (gel molecular weight ranges from 1,000 g/mol to 40,000,000 g/mol) having a length of 600 mm and an internal diameter of 7.5 mm and the guard column is a PL gel 20 μm, 50 mm length, 7.5 mm ID. The column temperature is 55° C. and the injection volume is 200 μL. The detector is a DAWN® Enhanced Optical System (EOS) including Astra® software, Version 4.73.04 detector software, manufactured by Wyatt Technology of Santa Barbara, Calif., USA, laser-light scattering detector with K5 cell and 690 nm laser. Gain on odd numbered detectors set at 101. Gain on even numbered detectors set to 20.9. Wyatt Technology's Optilab® differential refractometer set at 50° C. Gain set at 10. The mobile phase is HPLC grade dimethylsulfoxide with 0.1% w/v LiBr and the mobile phase flow rate is 1 mL/min, isocratic. The run time is 30 minutes.

A sample is prepared by dissolving the material in the mobile phase at nominally 3 mg of material/1 mL of mobile phase. The sample is capped and then stirred for about 5 minutes using a magnetic stirrer. The sample is then placed in an 85° C. convection oven for 60 minutes. The sample is then allowed to cool undisturbed to room temperature. The sample is then filtered through a 5 μm Nylon membrane, type Spartan-25, manufactured by Schleicher & Schuell, of Keene, N.H., USA, into a 5 milliliter (mL) autosampler vial using a 5 mL syringe.

For each series of samples measured (3 or more samples of a material), a blank sample of solvent is injected onto the column Then a check sample is prepared in a manner similar to that related to the samples described above. The check sample comprises 2 mg/mL of pullulan (Polymer Laboratories) having a weight average molecular weight of 47,300 g/mol. The check sample is analyzed prior to analyzing each set of samples. Tests on the blank sample, check sample, and material test samples are run in duplicate. The final run is a run of the blank sample. The light scattering detector and differential refractometer is run in accordance with the “Dawn EOS Light Scattering Instrument Hardware Manual” and “Optilab® DSP Interferometric Refractometer Hardware Manual,” both manufactured by Wyatt Technology Corp., of Santa Barbara, Calif., USA, and both incorporated herein by reference.

The weight average molecular weight of the sample is calculated using the detector software. A dn/dc (differential change of refractive index with concentration) value of 0.066 is used. The baselines for laser light detectors and the refractive index detector are corrected to remove the contributions from the detector dark current and solvent scattering. If a laser light detector signal is saturated or shows excessive noise, it is not used in the calculation of the molecular mass. The regions for the molecular weight characterization are selected such that both the signals for the 90° detector for the laser-light scattering and refractive index are greater than 3 times their respective baseline noise levels. Typically, the high molecular weight side of the chromatogram is limited by the refractive index signal and the low molecular weight side is limited by the laser light signal.

The weight average molecular weight can be calculated using a “first order Zimm plot” as defined in the detector software. If the weight average molecular weight of the sample is greater than 1,000,000 g/mol, both the first and second order Zimm plots are calculated, and the result with the least error from a regression fit is used to calculate the molecular mass. The reported weight average molecular weight is the average of the two runs of the material test sample.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for making an oral care article, the process comprising the steps of:
a. providing one or more soluble filament-forming materials;
b. forming an aqueous composition comprising the one or more soluble filament-forming materials;
c. processing the aqueous composition to produce a filament-forming composition;
d. delivering the filament-forming composition to one or more dies;
e. spinning the filament-forming composition to form a plurality of soluble filaments;
f. collecting the soluble filaments on a collection device to form a fibrous composition;
g. applying pressure to the fibrous composition to form a depression in the fibrous composition; and
h. adding a nonfibrous composition to the depression in the fibrous structure, wherein the nonfibrous composition is greater in weight percentage, by weight of the oral care article, than the fibrous composition.

2. The process of claim 1, wherein the process further comprises one or more converting operations selected from the group consisting of: slitting, stacking, calendering, treating with optional ingredients, die cutting, printing, packaging, and combinations thereof.

3. The process according to claim 1, wherein the one or more soluble filament-forming materials comprises pullulan, hydroxypropylmethyl cellulose, hydroxyethyl cellulose, methyl cellulose, hydroxypropyl cellulose, polyvinyl pyrrolidone, carboxymethyl cellulose, sodium alginate, xanthan gum, tragacanth gum, guar gum, acacia gum, Arabic gum, polyacrylic acid, methylmethacrylate copolymer, carboxyvinyl polymer, dextrin, pectin, chitin, levan, elsinan, collagen, gelatin, zein, gluten, soy protein, casein, polyvinyl alcohol, carboxylated polyvinyl alcohol, sulfonated polyvinyl alcohol, starch, starch derivatives, hemicellulose, hemicellulose derivatives, proteins, chitosan, chitosan derivatives, polyethylene glycol, tetramethylene ether glycol, hydroxymethyl cellulose, polyethylene oxide, or combinations thereof.

4. The process according to claim 3, wherein the polymer comprises polyvinyl alcohol, starch, or combinations.

5. The process according to claim 1, wherein up to about 60% by weight of water is added to the one or more filament-forming materials in step b.

6. The process according to claim 1, wherein the step of processing the aqueous composition occurs within an extruder.

7. The process according to claim 6, wherein the extruder exhibits a maximum screw speed of less than 1600 rpm.

8. The process according to claim 6, wherein the extruder exhibits a dry throughput of at least 2 kg/hr.

9. The process according to claim 6, wherein the extruder exhibits a wet throughput of at least about 5 kg/hr.

10. The process according to claim 6, wherein the filament-forming composition exits the extruder at an exit pressure of from about 10 to about 80 bar.

11. The process according to claim 1 wherein at least one of the soluble filaments comprises oral care active agent.

12. The process according to claim 11, wherein the oral care active agent comprises surfactant.

13. The process of claim 12, wherein the surfactant comprises anionic surfactant, cationic surfactant, nonionic surfactant, zwitterionic surfactant, amphoteric surfactant, or combinations thereof.

14. The process of claim 1, wherein the oral care active agent is added in the process in at least one of steps b, c, d, and h.

15. The process of claim 14, wherein the oral care active agent comprises fluoride ion source, metal ion source, abrasive, calcium ion source, polyphosphate, polymer, or combinations thereof.

16. The process of claim 1, wherein the nonfibrous composition comprises oral care active.

17. The process of claim 16, wherein the oral care active agents comprises fluoride ion source, metal ion source, abrasive, calcium ion source, polyphosphate, polymer, flavor, whitening agent, coolant, or combinations thereof.

18. The process of claim 1, wherein the depression is formed by a raised member on the collection device, by a raised member on a die cutter, or combinations thereof.
